(19) United States
(12) Patent Application Publication
(10) Pub. No.: US 2021/0052416 A1
(43) Pub. Date: Feb. 25, 2021

(54) GLAUCOMA TREATMENT BY DILATION OF COLLECTOR CHANNELS AND OSTIA

(71) Applicant: ALeyeGN Technologies LLC, Saratoga, CA (US)

(72) Inventors: Satish V. Herekar, Palo Alto, CA (US); John Randall Samples, Olympia, WA (US)

(73) Assignee: ALeyeGN Technologies LLC, Saratoga, CA (US)

(21) Appl. No.: 16/979,054

(22) PCT Filed: Mar. 8, 2019
(86) PCT No.: PCT/US2019/021431

§ 371 Date: Sep. 8, 2020

Related U.S. Application Data
(60) Provisional application No. 62/640,502, filed on Mar. 8, 2018.

(51) Int. Cl. A61F 7/00

(52) U.S. Cl. CPC A61F 7/00; A61N 5/0625; A61F 2007/0004

(57) ABSTRACT

Methods, systems, and apparatus to treat glaucoma of the eye are disclosed. They apply energy to the tissue adjacent collector channels and/or Schlemm's Canal. Applying energy to the surface of the eye, such as the sclera, adjacent the collector channels and/or their ostia can dilate the collector channels and/or their ostia to improve uveoscleral outflow to reduce intraocular pressure. In some examples energy is applied to the eye with a laser at a sufficient energy to shrink the tissue on opposing sides of the collector channels to induce channel dilation.

### CROSS REFERENCE TO RELATED APPLICATIONS

This is a non-provisional application of U.S. 62/640,502 filed Mar. 8, 2018. The contents of that provisional application are incorporated herein by reference.

The subject matter of the present application is related to the following patent applications, the entire disclosures of which are incorporated by reference herein to the extent they are not inconsistent with the present disclosure:

U.S. App. Ser. No. 62/385,234, filed Sep. 8, 2016, entitled “EFFECTIVE OCULAR LENS POSITIONING AND GLAUCOMA TREATMENT METHODS AND APPARATUS,” U.S. App. Ser. No. 62/473,269, filed Mar. 17, 2017, entitled “GLAUCOMA TREATMENT METHODS AND APPARATUS,” U.S. App. Ser. No. 62/556,228, entitled “GLAUCOMA TREATMENT METHODS AND APPARATUS,” PCT/US2017/50799, filed Sep. 8, 2017, entitled “GLAUCOMA TREATMENT METHODS AND APPARATUS,” PCT/US2017/023092, filed on 17 Mar. 2017, entitled “EFFECTIVE OCULAR LENS POSITIONING METHODS AND APPARATUS,” PCT/US2016/055829, filed on 6 Oct. 2016, entitled “ULTRASOUND DIRECTED CAVITATIONAL METHODS AND SYSTEMS FOR OCULAR TREATMENTS,” PCT/US2014/023763, filed 11 Mar. 2014, entitled “SCLERAL TRANSLOCATION ELASTO-MODULATION METHODS AND APPARATUS” U.S. Provisional Application 62/561,642, filed 21 Sep. 2017, entitled “ANGLE OPENING GLAUCOMA TREATMENT METHODS AND APPARATUS” and PCT/US2018/052261, filed 21 Sep. 2018, entitled “ANGLE OPENING GLAUCOMA TREATMENT METHODS AND APPARATUS.”

All publications, patents, and patent applications mentioned in this specification are herein incorporated by reference to the same extent as if each individual publication, patent, or patent application was specifically and individually indicated to be incorporated by reference.

### BACKGROUND

Glaucoma is a group of diseases characterized by increased intraocular pressure (IOP) that result in optic nerve damage. Aqueous humor is produced from the ciliary processes, moves through the pupil into the anterior chamber and into the trabecular meshwork, Schlemm's canal, and uveoscleral outflow pathways. Increased IOP results from an imbalance between the production of aqueous humor from the ciliary body and resistance to its outflow through the normal anatomic outflow tract. Glaucoma can lead to progressive deterioration of the optic nerve associated with cupping and atrophy of the optic disc. The effects of this damage are accompanied by a progressive loss of the peripheral visual field followed by a loss of central vision that results in irreversible blindness if not timely treated.

The anterior chamber of the eye is the aqueous humor-filled space between the iris and the cornea's innermost surface. The “angle” of the anterior chamber refers to the angle between the iris and the cornea (iridocorneal angle) that is near the limbus which circumscribes the cornea at the border between the transparent cornea and the opaque sclera. Near the vertex of the iridocorneal angle is the trabecular meshwork and Schlemm's canal through which much of the aqueous humor leaves the eye to maintain normal IOP. The depth of the anterior chamber varies between 1.5 and 4.0 mm, averaging 3 mm, and it tends to become shallower with age. Although there are many causes of glaucoma, the most common types are defined with reference to the angle of the anterior chamber: open-angle glaucoma and angle closure glaucoma. Open-angle glaucoma usually develops slowly and painlessly over time and is commonly attributed to a functional or structural obstruction of aqueous outflow within the trabecular meshwork or uveoscleral tract. Angle closure glaucoma (also known as narrow-angle glaucoma) typically occurs when the iris moves forward and narrows the angle of the anterior chamber between the iris and cornea to decrease the depth of the anterior chamber. Angle closure glaucoma can present gradually or suddenly. The sudden presentation may involve eye pain, blurred vision, dilation of the pupil, hyperemia and even nausea. Although these diseases have been extensively studied their causes are not completely understood.

The most common treatment for glaucoma is the use of medication such as eye drops. Although these medications have greatly improved glaucoma treatment, topical medication in the eye potentially causes local and systemic side-effects. Patient adherence can also be unpredictable and life-long use of the medication can be expensive. Poor compliance with medication use over extended periods of time is a major reason for vision loss in glaucoma patients. To help avoid these problems, and treat refractory cases, surgical interventions such as trabeculotomies and antifibrotics with tube shunts have been developed. However, surgical glaucoma treatments are complex and invasive.

Although lasers, stents and ultrasound have been proposed to promote the flow of fluid from the anterior chamber of the eye into Schlemm's canal, they may not adequately treat glaucoma in at least some patients. There may also be many mechanisms related to increased intraocular pressure, and even if one mechanism is adequately addressed another mechanism may perpetuate the problem.

Improved methods and apparatus of treating glaucoma are needed. Ideally, such methods and apparatus would be less invasive than some prior treatments and provide successful reduction in IOP, even for treatment-resistant cases.

### SUMMARY

According to an example of the disclosed technology, systems for treating glaucoma of an eye can include a processor configured with instructions to receive input corresponding to a plurality of locations of collector channels coupled to a Schlemm's canal of the eye, and generate a plurality of treatment locations for the eye in response to the plurality of locations, wherein the treatment locations are adjacent one or more collector channels and are spaced laterally from the collector channels by a distance of no more than 1 mm, and include an energy source configured to generate energy to treat the eye, and a scanner operably coupled to the energy source and the processor, the scanner configured to deliver the energy to the plurality of treatment locations to shrink tissue at the treatment locations and dilate the one or more of collector channels of the eye or ostia of the collector channels. In some examples, the plurality of treatment locations include pairs of opposing treatment locations situated on opposite sides of each of the one or more collector channels. At least one of the pairs of treatment locations can be positionally configured to stretch tissue between the opposing treatment locations of the at least one pair to provide the dilating of the one or more collector channels to increase flow of the collector channels of the eye. In some examples, the processor is configured with instructions to identify the one or more collector channels or ostia from image data of the eye. The identification can include using a trained convolutional neural network configured to identify patterns in one or more sets of optical coherence tomography slices proximate Schlemm's canal and the collector channels. In some examples, the processor is configured with instructions to repeatedly deliver the energy to each of the plurality of treatment locations with a time delay in order to fractionate delivery of energy to each of the plurality of treatment locations. The time delay can be within a range from about 10 millisecond (ms) to about 60 (s) and optionally wherein the time delay is within a range from about 100 ms to about 30 s and optionally within a range from about 500 ms to about 15 s and optionally within a range from about 1 to about 10 s. In selected examples, the processor is coupled to the energy source and the scanner and is configured with instructions to heat tissue at the plurality of treatment locations to a temperature within a range from 50 to 70 (° C.) at a depth within a range from 50 to 400 μm. The plurality of treatment locations can extend in a treatment pattern arranged to avoid or reduce a heating of tissue overlaying one or more of the Schlemm's canal or at least one of the collector channels to the Schlemm's canal. In different examples, the energy source includes one or more of a pulsed laser, a continuous wave laser, a pulsed ultrasound transducer, a HIFU array, or a phased HIFU array. The input can include an input from a user of the system or an input from an imaging apparatus. In some examples, the energy source includes a laser having a wavelength within a range from about 0.8 to 2.3 μm. The energy source can be configured to generate a treatment spot at or in the eye, the treatment spot being in a range of 30 to 500 μm across. In some examples, the energy source can be configured to generate an average power of 200 mW to 1400 mW.

According to another aspect of the disclosed technology, systems to treat glaucoma of an eye can include an energy source, and a handpiece coupled to the energy source and including an eye contacting surface to couple to the eye and a plurality of energy releasing elements disposed at a plurality of locations to direct energy to the eye to a plurality of treatment locations, wherein each treatment location is adjacently spaced apart by a distance of no more than 1 mm from a collector channel coupled to a Schlemm's canal of the eye, wherein the positions of the treatment locations and the energy directed to the treatment locations are configured to shrink tissue at the treatment locations to produce a dilation of the adjacent collector channels or ostia of the collector channels. In some examples, the plurality of treatment locations include pairs of opposing treatment locations, with a first treatment location of each pair situated at a first position adjacent to one of the collector channels and a second treatment location of each pair situated at a second position adjacent to the one collector channel and opposite the first position. The plurality of energy releasing elements can include a plurality of optical fibers and the energy source includes a laser. In additional examples, the plurality of energy releasing elements include a plurality of electrodes and the energy source includes an electroporation energy source, a microwave energy source, a thermal energy source, an electrical energy source, an electrophoretic energy source, or a di-electrophoretic energy source. Representative examples further include a processor coupled to the energy source to control delivery of the energy to the plurality of treatment locations and optionally to fractionate energy delivered to each of the plurality of treatment locations.

According to a further aspect of the disclosed technology, methods for treating glaucoma of an eye include determining a plurality of locations of collector channels coupled to a Schlemm's canal of the eye, and delivering energy to a plurality of treatment locations adjacent to collector channels of the eye based on the plurality of locations, wherein the treatment locations are located within 1 mm laterally of the collector channels, wherein the energy is delivered to the plurality of treatment locations to shrink tissue at the treatment locations to stretch one or more of at least one collector channel or an ostia of the at least one collector channel In some examples, the plurality of treatment locations includes pairs of opposing treatment locations situated on opposite sides of each of the at least one collector channels to produce the stretching between opposing treatment locations to produce a dilation of the collector channel straddled by the opposing treatment locations or ostium of the straddled collector channel. At least one of the treatment locations can correspond to an opposing treatment location of two different pairs. In representative examples, the tissue is heated to a temperature within a range from 50 to 70° C. at a depth within a range from 50 to 400 μm at each of the treatment locations. In some examples, the determining the locations includes identifying the collector channels from optical coherence tomography image data of the eye. In some examples, the plurality of treatment locations is arranged to minimize shrinking of tissue overlaying one or more of the collector channels or the Schlemm's canal. The energy can be delivered from one or more of a pulsed laser, a continuous wave laser, a pulsed ultrasound transducer, a HIFU array, or a phased HIFU array. The energy can be delivered from a laser having a wavelength within a range from about 0.8 to 2.3 μm. In some examples, the energy is configured to generate a treatment spot in the eye, the treatment spot being in a range of 30 to 500 μm across.

According to another aspect of the disclosed technology, apparatus to treat glaucoma of an eye having a Schlemm's canal and collector channels coupled thereto, include an energy source and a processor coupled to the energy source, wherein the processor is configured with instructions to direct energy in an irregular pattern associated with an irregular azimuthal positioning of the collector channels to shrink collagenous tissue near the collector channels coupled to the Schlemm's canal to dilate the collector channels. In representative examples, the energy source includes a laser, such as a laser having wavelength within a range from about 0.8 um to about 2.1 um. In some examples, the energy source is configured to deliver an amount of energy per unit time (power) to the eye within a range from about 50 mW to about 900 mW, preferably within a range from about 100 mW to about 700 mW, more preferably within a range from about 200 to 400 mW. In different examples, the energy source can include one or more of a pulsed laser, a continuous wave laser, a pulsed ultrasound transducer, a HIFU array, or a phased HIFU array. In some examples, the processor is configured with instructions to apply a total amount of energy applied to the eye to treat glaucoma within a range from about 4 J to about 90 J, preferably within a range from about 5 J to about 50 J, with a treatment time within a range from about 4 to 200 seconds, preferably within a range from about 8 to 100 seconds and optionally the energy source can include an ultrasound energy source or a laser. In some examples, the processor is configured with instructions to scan the energy source to the treatment locations on opposites side of the collector channels with a scan rate within a range from about 10 to 100 mm/second, preferably within a range from about 12 to 50 mm/s, more preferably within a range from about 20-30 mm/s, for example, about 25 mm/s and optionally wherein the energy source includes an ultrasound energy source or a laser. In selected examples, the energy source includes a laser that produces a cross-sectional beam spot size at or in the eye within a range from about 30 to 500 μm, preferably within a range from about 150-400 μm, more preferably within a range from about 200-300 μm spot size. In some examples, the energy source includes an ultrasound transducer. In representative examples, the irregular pattern is an irregular annular pattern. In some examples, the processor is configured with instructions to identify collector channels and/or ostia from OCT images of the eye. In typical examples, the energy source includes an optical scanner, and the processor is configured with instructions to direct the energy to the treatment locations using the optical scanner. Some examples further include a lens having a concavely curved surface to contact the eye and conduct heat from tissue heated with the energy source.

According to another aspect of the disclosed technology, a system for treating an eye (the eye including a Schlemm's canal and collector channels coupled thereto) includes a processor, configured with instructions to receive an anterior image of the eye generated with a camera anterior to the eye, estimate a plurality of collector channel locations in response to the anterior image of the eye or a plurality of optical coherence tomography (OCT) images of the eye associated with the anterior image, determine a plurality of treatment locations for the eye in response to the plurality of the collector channel locations, and overlay of the plurality of treatment locations and the plurality of collector channel locations on the anterior image of the eye shown on a display.

According to a further aspect of the disclosed technology, systems for treating an eye include a processor configured with instructions to estimate a plurality of locations of collector channels of the eye, the collector channels coupled to a Schlemm's canal of the eye, and generate a plurality of treatment locations for the eye in response to the plurality of locations, wherein each of the treatment locations is spaced laterally from adjacent one of the collector channels by a distance of no more than 1 mm, and includes an energy source configured to generate energy for treating the eye and a scanner operably coupled to the energy source and the processor, the scanner configured to deliver the energy to the plurality of treatment locations to dilate the one or more of collector channels of the eye or ostia of the collector channels.

According to an additional aspect of the disclosed technology, methods for treating an eye (the eye including a Schlemm's canal and collector channels coupled thereto) include receiving, with a processor, an anterior image of the eye generated with a camera anterior to the eye, estimating, with the processor, a plurality of collector channel locations in response to the anterior image of the eye or a plurality of optical coherence tomography (OCT) images of the eye, determining, with the processor, a plurality of treatment locations for the eye based on the plurality of collector channel locations, and overlaying, with the processor, the plurality of treatment locations and the plurality of collector channel locations on the anterior image of the eye. In some examples, the processor is further configured to register the plurality of locations of collector channels with a corresponding plurality of anterior image locations. In representative examples, the plurality of treatment locations includes pairs of opposing treatment locations situated on opposite sides of each of the collector channels, wherein each pair is positionally configured to stretch tissue between the opposing treatment locations of the pair to provide the dilating of the collector channels to increase flow of the collector channels of the eye. In some examples, the plurality of treatment locations includes a first plurality of treatment locations positioned on a first side of each of the collector channels locations and a second plurality of treatment locations on a second side of each of the collector channels opposite the first side. In selected examples, the processor is configured with instructions to alternate treatment at the first plurality of treatment locations with treatment at the second plurality of treatment locations. In some alternating examples, alternating the treatment between the first plurality of treatment locations and the second plurality treatment locations decreases movement of tissue at a location between the first plurality of treatment locations and the second plurality of treatment locations. In further alternating examples, the alternating the treatment between the first plurality of treatment locations and the second plurality treatment inhibits biasing of tissue at the first plurality of treatment locations or the second plurality of treatment locations. Some examples further include delivering energy from an energy source in a treatment sequence alternating between a treatment location of the first plurality of treatment locations and a treatment location of the second plurality of treatment locations along a scan path defined by successive distances between each of the plurality of treatment locations and an immediately prior treatment location of the sequence. In some examples, scan paths can include a circular, an annular, an oval, or an elliptical path or a portion thereof. In some examples, each of the plurality of treatment locations corresponds to a location on the anterior image and wherein the processor is configured with instructions to generate a treatment table, the treatment table including a plurality of coordinate reference locations corresponding to the plurality of treatment locations overlaid on the anterior image and optionally wherein the energy source includes a pulsed energy source and wherein each of the plurality of coordinate references corresponds to a pulse from an energy source. In some examples, the collector channel locations are estimated based on a plurality of limbus locations of the eye, and in selected examples, the plurality of limbus locations is determined in response to changes in intensity of the anterior image. Some examples further include generating the anterior image of the eye with a camera, and some camera examples can include a video camera configured to capture images of the eye and the processor is configured to overlay the plurality of treatment locations on the images shown on the display in real-time. Some examples further include delivering energy with an energy source to the treatment location in accordance with a user's instructions. In representative examples, the energy source includes a laser, such as a laser configured to generate a first light beam having a wavelength within a first range from about 1.4 to 1.6 μm and second light beam having a second wavelength within a range from about 1.9 to 2.3 μm. In some laser examples, the laser is configured to generate a first light beam having a wavelength of about 1.47 μm and second light beam having a second wavelength of about 2.01 μm. Some examples further include delivering the energy with the energy source using a scanner operably coupled to the energy source and configured to deliver the energy to the plurality of treatment locations. In representative examples, each of the plurality of OCT images includes a slice along a plane of tissue of the eye and wherein each of the plurality of slices is registered with the eye to determine the plurality of locations of collector channels coupled to the Schlemm's canal along a two-dimensional path and optionally wherein each of said slices is rotated about an optical axis of the eye with respect to other slices around the optical axis of the eye. In some examples, a 2D treatment pattern projected onto the eye includes a plurality of locations on either side of each of the collector channels on anterior layer of the eye or facing ostia of said each of the collector channels, the anterior layer selected from the group consisting of a cornea of the eye and a sclera of the eye.

According to another aspect of the disclosed technology, systems for treating glaucoma of an eye include a processor configured with instructions to generate a plurality of treatment locations for the eye, wherein the plurality of treatment location is located within 1 mm of collector channels coupled to the Schlemm's canal, the plurality of treatment locations being opposing tissue locations spaced apart laterally from the collector channels by a distance of no more than 1 mm, include an energy source configured to generate energy to treat the eye, and include a scanner operably coupled to the energy source and the processor, the scanner configured to deliver the energy to the plurality of treatment locations in order to dilate the one or more of collector channels of the eye or ostia of the collector channels.

According to a further aspect of the disclosed technology, systems to treat glaucoma of an eye include an energy source, a handpiece coupled to the energy source, the handpiece including an eye contacting surface to couple to the eye, and a plurality of energy releasing elements disposed at a plurality of locations to release energy to the eye at a plurality of treatment locations, wherein the plurality of locations corresponds to opposite treatment locations spaced apart by no more than 1 mm laterally from the collector channels coupled to the Schlemm's canal of the eye.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates an eye.

FIG. 2 illustrates stabilization of an eye by cross-linking to treat presbyopia.

FIG. 3 illustrates common fluid outflow paths of the eye including the location of the limbus and Schlemm's Canal.

FIG. 4 illustrates an ostia/collector channel adjustment system for treating an eye.

FIG. 5 shows a schematic of a treatment system.

FIGS. 6A-6C show an embodiment of a handheld probe.

FIG. 7 illustrates a heat sink placed over the eye of FIG. 2.

FIGS. 8A-8C show a structure for coupling an energy source to a surface of an eye.

FIG. 9 shows temperature profiles of an eye treated with a laser beam with the eye coupled to a chilled lens.

FIG. 10 shows a treatment system for ostia and/or collector channel adjustment.

FIG. 11 shows a ostia/collector channel adjustment system.

FIG. 12 shows a HIFU array coupled to an imaging apparatus.

FIG. 13 shows another HIFU array coupled to an imaging apparatus.

FIG. 14 shows a schematic of a one-dimensional HIFU system.

FIG. 15A shows an anterior view of the trabecular meshwork of the eye and adjacent Schlemm's Canal and ostia that communicate with the collector channels.

FIG. 15B shows a magnified section view the Schlemm's canal of the eye.

FIG. 16 shows a treatment user interface for treating the eye.

FIG. 17 shows a schematic diagram of treatment locations within the eye adjacent to and on opposing sides of collector channels.

FIG. 18 shows a treatment user interface for treating the eye.

FIG. 19 shows a flowchart of a method for determining target treatment locations and treating the eye.

FIG. 20 is a perspective view depicting OCT generated image slices of an eye.

FIG. 21 is a flowchart of a method of identifying and treating collector channels.

FIG. 22 is a flowchart showing training and use of a convolutional neural network.

FIG. 23 is a schematic of a computing environment for imaging and treating an eye.

FIGS. 24A & 24B are anterior views depicting an opening of ostia and collector channels coupled to Schlemm's canal with selective laser treatment.

### DETAILED DESCRIPTION

### Terms

As used in this application and in the claims, the singular forms “a,” “an,” and “the” include the plural forms unless the context clearly dictates otherwise. Additionally, the term “includes” means “comprises.” Further, the term “coupled” does not exclude the presence of intermediate elements between the coupled items.

The systems, apparatus, and methods described herein should not be construed as limiting in any way. Instead, the present disclosure is directed toward all novel and non-obvious features and aspects of the various disclosed embodiments, alone and in various combinations and sub-combinations with one another. The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Although the operations of some of the disclosed methods are described in a particular sequence for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show various ways in which the disclosed systems, methods, and apparatus can be used in conjunction with other systems, methods, and apparatus.

With reference to eye anatomy, “anterior” refers to the front of the eye, toward the anterior pole. “Posterior” refers to the back of the eye, toward the posterior pole. “Lateral” refers to being spaced from the sides of a reference structure, such as a collector channel “Nasal” refers to a direction toward the nose, and “temporal” refers to direction toward the temple. Collector channels extend posteriorly from Schlemm's Canal on the curved surface of the eye, and locations along the outer edges of the collector channels are lateral to the collector channels.

Tissue adjustment procedures apply heat to the eye to produce a thermo-mechanical response in a target tissue of the eye, such as in the cornea and/or sclera. Examples of these procedures include scleral translocation elasto modulation (“STEM”). For example, the cornea and/or sclera can be heated to a range from about 50 to about 70 degrees Centigrade, for example between 60 and 70 degrees Centigrade, to produce shrinkage of the tissue. Tissue may be heated within the range without substantially weakening the tissue. In some embodiments, a portion of the eye can be heated to a temperature within a range of up to about 55 or 60 degrees Centigrade to relax the tissue. Heating the cornea and/or sclera to a temperature within this range can produce softening and/or plasticizing of the tissue (e.g., to approximately 10% of the native strength of the tissue). The cornea and/or sclera can be heated to greater than 80 degrees Centigrade to produce denaturation of the tissue. The tissue may be weakened by heating to a temperature within a range from about 70 to about 90 degrees Centigrade.

The heating of tissue can be controlled to provide desired amounts of shrinkage or relaxation and combinations thereof. For example, heating collagenous tissue such as scleral tissue to a temperature within a range from about 50 to 70 degrees C. can result in shrinkage of the tissue that can be effective to move tissue to open Schlemm's canal and/or the collector channels and/or their ostia coupled thereto for example. For temperatures in a range from about 60 to 70 degrees C., heating of the tissue can result in shrinkage or relaxation, depending on how long the tissue is heated. For example, heating tissue within a range from 60 to 70 degrees C. for shorter amounts of time can result in tissue shrinkage, while heating tissue for longer amounts of time can result in relaxation. To relax tissue, the tissue temperature can be elevated to a temperature in a range from about 60 degrees C. to about 80 degrees C. For example, heating tissue to about 80 degrees C. for about a millisecond can result in tissue relaxation. For lower temperatures within this 60 to 80 C range, the tissue can be heated for amounts of time longer than 1 ms to provide tissue relaxation.

When light energy is used, the depth of tissue with sufficient heating can depend on the wavelengths of light energy. For example, light energy having wavelengths in a range from about 1.9 to 2.1 um, the 1/e attenuation depth can be in a range from about 200 to 300 um, for example about 225 to 275 um. For light energy having wavelength in a range from about 1.3 to 1.6 um, the 1/e attenuation depth is within a range from about 350 to 450 um. When combined with the cooling lens as described herein, the profile of tissue heating can result in a peak temperature that is located beneath the exterior surface of the ocular tissue, even though the amount of light energy absorbed near the surface is greater than the amount of light energy absorbed at the tissue location which undergoes the highest amount of temperature increase. The depth of tissue that shrinks or relaxes can have a profile extending to a depth in the tissue.

The ultrasound methods and apparatus disclosed herein can be used to heat tissue with similar temperatures and locations as described with reference to lasers for treating glaucoma as described herein. Other types of energy can alternatively be used to treat glaucoma as will be appreciated by one of ordinary skill in the art. The ultrasonic approaches can also be used to soften tissue without substantial heating, for example.

In many embodiments, the methods and apparatus can be used to treat one or more of many disorders of the eye with an energy source, under control of computer instructions. The apparatus can be used to shrink tissue adjacent to the collector channels or ostia to enlarge them and increase flow of intraocular fluid through them. For example the methods and apparatus can be used in a thermal mode to increase the temperature of the treated tissue to more than about 50 degrees C., for example about 60 degrees C. or more. The non-thermal treatment can be used in many ways, such as for accurate tissue resection. Alternatively, adjustment-induced ultrasonic cavitation can focally disrupt or liquefy or micro-porate (spongify) tissue and reduce rigidity, thus enhancing mobility of accommodative complexes and/or aqueous outflow facilities. In some examples tissue is tightened adjacent the collector channels and ostia and tissue is mobilized further from the collector channels and ostia to enhance constriction of tissue at the desired constriction locations and dilation of the collector channel.

### Light Energy Sources

Collector channel and/or ostia adjustment system examples may include an energy delivery system configured to deliver energy to the eye. In some examples the target tissue is the sclera or the cornea, and more preferably locations proximate collector channels and/or ostia with energy directed through the sclera and not the cornea. One or more of the energy source, processor, or energy delivery system may be configured to deliver energy to the eye.

In many embodiments, the adjustment procedures provide extra-corneal and/or extra-lenticular energy treatment to soften and/or plasticize the sclera and/or peripheral cornea, such as with one or more of light energy, ultrasound energy, high intensity ultrasound energy, mechanical energy, radiofrequency energy, electrical energy, thermal energy, electroporation, microwave energy, optoporation, photonic desincrustation, or galvanic desincrustation. These methods are disclosed in U.S Patent Publication 2018/0207029 which is incorporated by reference herein. Any of the energy sources described in that application may be used when plasticization of eye tissue is desired.

The energy can be delivered with an optical delivery system, for example from a hand-held probe or a laser scanner.

In many embodiments, the light energy comprises wavelengths that are absorbed more strongly by stromal tissue than water, for example light comprising a wavelength within a range from about 4 to 6 micrometers (μm), such as from about 5.5 to 6.6 μm. The light energy absorbed more strongly by stroma than water has the advantage of providing more accurate treatment with less interference with water and can allow the tissues of the eye to retain healthy amounts of water during treatment, for example tissues of the conjunctiva of the eye. Also, interference from water based surgical fluids such as saline and anesthetics can be substantially inhibited.

In many embodiments, the light energy comprises wavelengths within a range from about 1 to 6 μm, such as from about 1 to 3 μm. In many embodiments the light energy comprises wavelengths within a range from about 1.4 to about 2 μm, for example about 1.46 μm or 2.01 μm, and other ranges as described herein.

The laser may comprise one or more of many lasers emitting one or more of many wavelengths, such as infrared lasers. In many embodiments, the laser comprises a quantum cascade laser configured to emit light having a wavelength within a range from about 5.8 to about 6.6 μm, for example from about 6 to about 6.25 μm. In many embodiments, the laser comprises a quantum cascade laser or continuous wave laser configured to emit light having a wavelength within a range from about 1 to about 6 μm, such as from about 1 to 3 μm. In many embodiments the laser is configured to emit light having a wavelength within a range from about 1.4 to about 2 μm, for example about 1.46 μm or 2.01 μm, and other wavelength ranges as described herein. Such lasers are commercially available and can be configured by a person of ordinary skill in the art for treatment of the eye as described herein.

The energy source may comprise one or more of a pulsed laser, a continuous wave (CW) laser, a pulsed ultrasound transducer, a HIFU array, or a phased HIFU array. The energy source may comprise an ultrasound transducer.

The plurality of energy releasing elements may comprise a plurality of optical fibers and the energy source may comprise a laser. The energy releasing elements may include electrodes and the energy source may include an electroporation energy source, a microwave energy source, a thermal energy source, an electrical energy source, an electrophoretic energy source, or a di-electrophoretic energy source. The system may further comprise a processor coupled to the energy source to deliver energy to the plurality of treatment locations and optionally wherein the processor may be configured to fractionate energy delivered to each of the plurality of treatment locations.

The processor may be configured with instructions to apply a total amount of energy applied to the eye to treat glaucoma within a range from about 4 J to about 90 J, preferably within a range from about 5 J to about 50 J, with a treatment time within a range from about 4 to 200 seconds (s), preferably within a range from about 8 to 100 s and optionally wherein the energy source may comprise an ultrasound energy source or a laser. The processor may be configured with instructions to scan the energy source along the eye with a scan rate within a range from about 10 to 100 mm/second, preferably within a range from about 12 to 50 mm/s, more preferably within a range from about 20-30 mm/s, for example, about 25 mm/s and optionally wherein the energy source may comprise an ultrasound energy source or a laser. The energy source may comprise a laser, for example having a cross-sectional beam spot size within a range from about 100 to 500 μm, preferably within a range from about 150-400 μm, more preferably within a range from about 200-300 μm spot size when applied to the tissue near the collector channel or ostia. The energy source may comprise a laser or an ultrasound energy source and the treatment may have a duration from about 8 to about 100 seconds.

The energy source may comprise a laser having a wavelength within a range from about 1.9 to 2.3 μm, such as about 1.9 μm. The energy source may be configured to generate a treatment spot in the eye, the treatment spot being in a range of 50 μm to 300 μm across, such as about 100 μm to 200 μm across.

The laser energy may have a wavelength within a range from about 1.5 μm to about 2.1 μm. The energy source may comprise an amount of energy per unit time (power) delivered to the surface of the eye within a range from about 50 mW to about 900 mW, preferably within a range from about 100 to about 700 mw, more preferably within a range from about 200 to 400 mW.

### Heat Sink and Spacer

In many embodiments a heat sink is coupled to the conjunctiva and is made of a material transmissive to the light energy, such as sapphire or Zinc Selenide (hereinafter “ZnSe”). The heat sink material can be configured to transmit light energy absorbed more strongly by the stroma than water and may comprise Zinc Selenide (hereinafter “ZnSe”), for example. The heat sink can be chilled to inhibit damage to the conjunctiva of the eye. The heat sink can provide improved transmission of light energy when condensation is present, as the condensed water may be less strongly absorbed by the laser beam. In many embodiments, one or more layers of the epithelium of the eye (basal layer, wing layer or squamous layer) remains substantially intact above the zone where the eye has been treated, for example at least one layer of viable epithelial cells can remain intact when the heat sink is removed.

In many embodiments, the optically transmissive material of the heat sink is shaped and optically configured with smooth surfaces to comprise an optically transparent heat sink such as a lens. The heat sink may comprise a window of the optically transmissive material and can be one or more of many shapes such as a flat on opposing surfaces, plano-concave, or convex-concave. In some examples the convex-concave heat sink window may comprise a meniscus shaped lens having substantial optical power or no substantial optical power.

The location of the heat sink can be fixed in relation to a fixed structure of the laser system to fix the location of the eye, and the heat sink may comprise one or more curved surfaces such as a concave surface to engage the eye. In many embodiments, an arm extends from the fixed structure of the laser system to the heat sink to fix the location of the heat sink.

In many embodiments the collector channel and ostia treatment apparatus includes an energy source such as a laser and a docking station to retain the eye in a target location. The docking station may include the chilled optically-transmissive heat sink to couple to the eye. The docking station couples to the eye such that the heat sink contacts the conjunctiva of the eye and fixes the working distance of the eye relative to the surgical laser, such that the scleral treatment can be performed accurately. In many embodiments, the heat sink is chilled such that at least one epithelial layer of the conjunctiva of the eye above the treated tissue remains viable, to expedite healing of the eye and decrease invasiveness of the procedure. The chilled heat sink structure can be chilled to a temperature within a range from above the freezing temperature of the eye and saline, at about −3 degrees Celsius (° C.), to below an ambient room temperature of about 20° C. Alternatively, a heat sink can be provided without chilling. In many embodiments, the freezing temperature of the eye corresponds to the freezing temperature of saline, about −3° C., for example. In many embodiments, the apparatus comprises a scanner to scan the laser beam. The laser beam can be pulsed or continuous, and in many embodiments comprises a continuous laser beam configured to inhibit temperature spikes related to ablation of the eye. In many embodiments the laser irradiance comprises a temporal and spatial profile to inhibit transient heating peaks of the tissue that can be related to removal of tissue such as ablation. The scanner can be configured to scan the laser beam in a plurality of quadrants, such as for quadrants with untreated regions between each of the quadrants to inhibit damage to muscles of the eye located between the treatment quadrants.

The cooling methods and apparatus disclosed herein can be combined with the energy sources described herein in order to decrease heating of tissue near external surfaces of the eye, such as conjunctival and epithelial layers of the eye. Decreased heating of tissue near external surfaces of the eye may result in the tissue near the external surfaces of the eye remaining substantially viable when the tissue below it is treated. This may for example be done in order to inhibit pain and swelling of the eye during and/or after treatment.

### Glaucoma

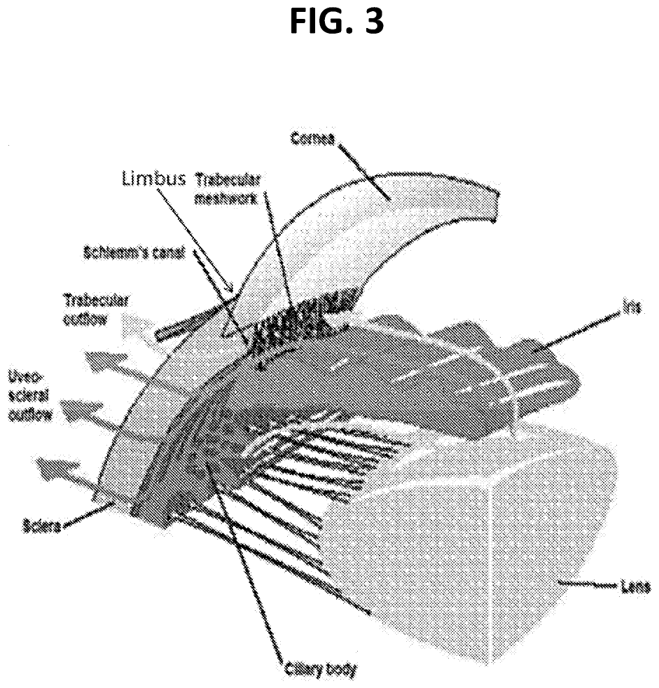

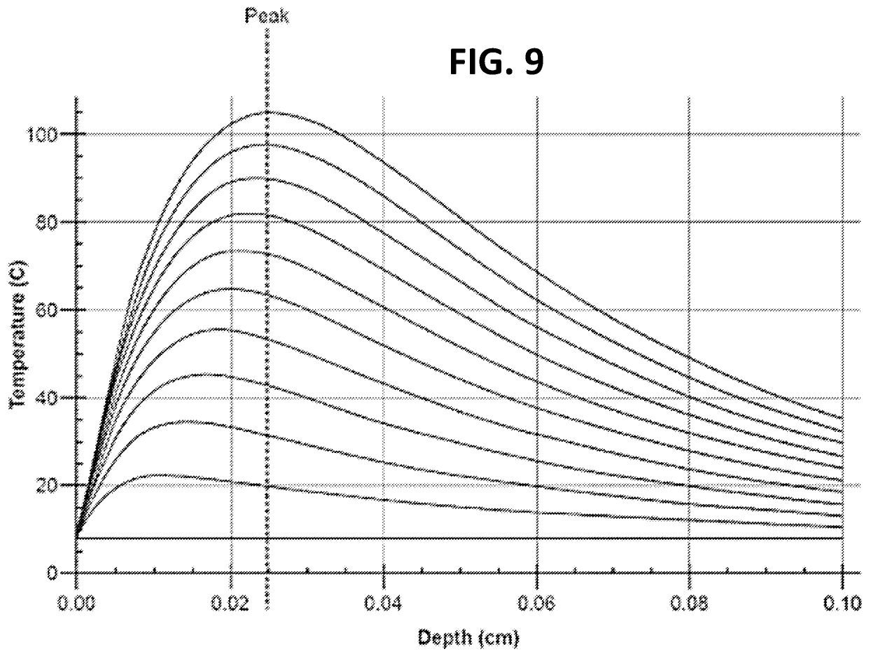

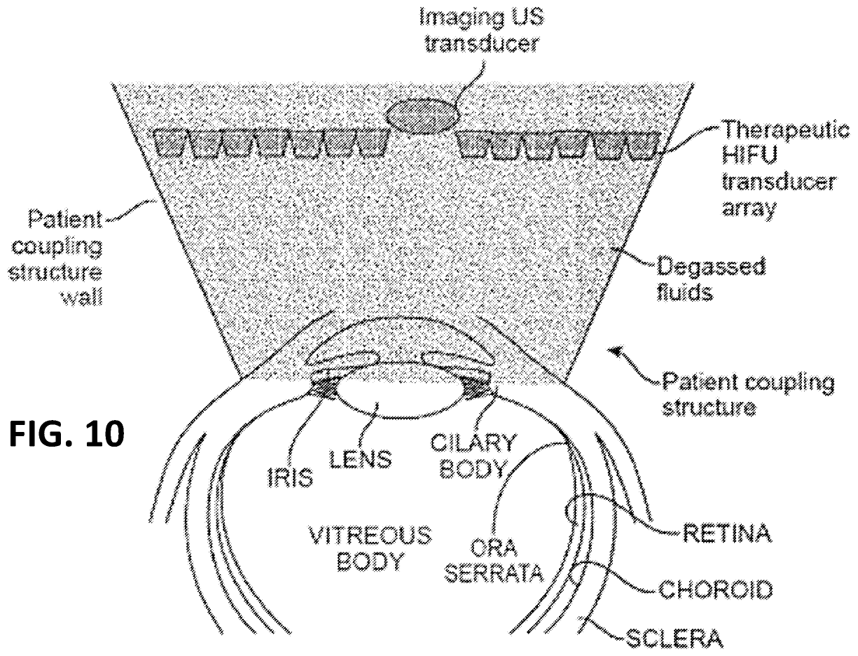

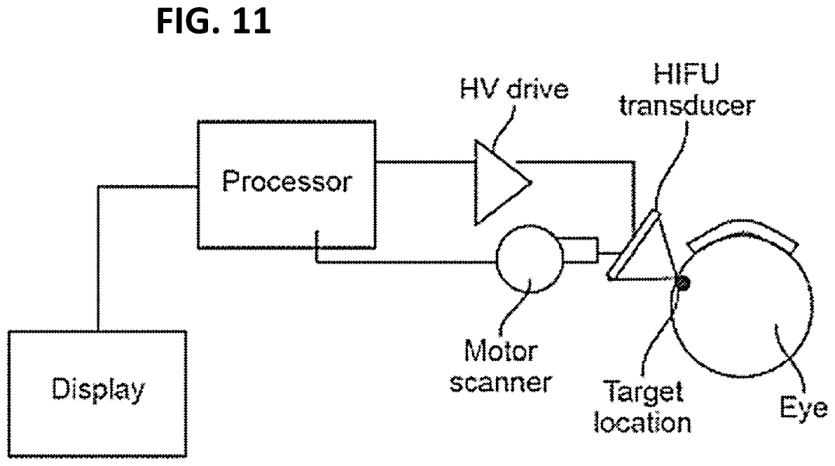

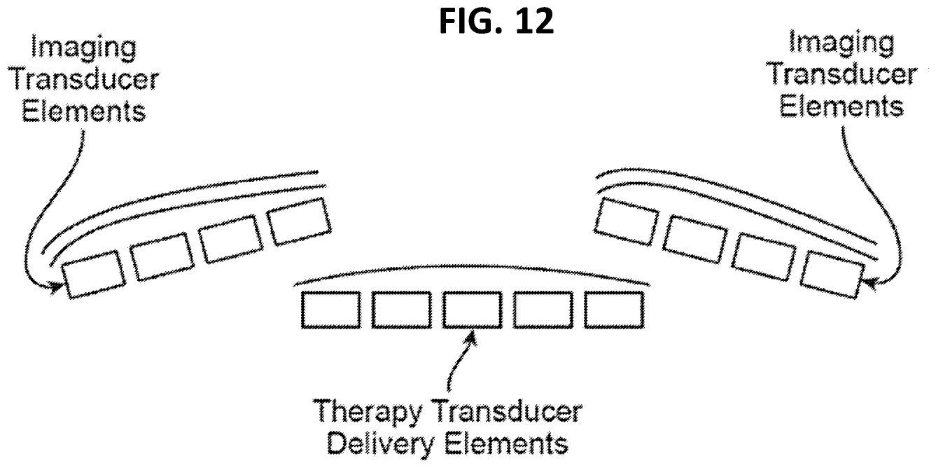

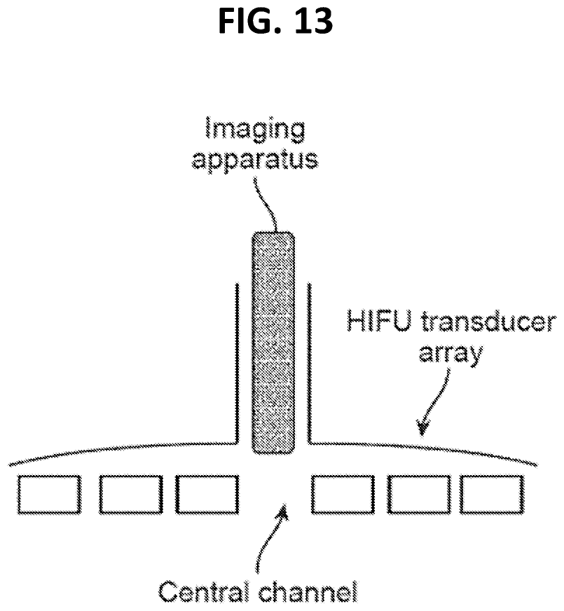

FIGS. 3-24 show glaucoma treatment methods and apparatus as will be understood by a person of ordinary skill in the art. The methods and apparatus as described herein can be combined in many ways to treat glaucoma, for example with reference to PCT/US2017/023092, the entire disclosure of which is hereby incorporated by reference, which may be combined with FIGS. 3-24 in accordance with embodiments disclosed herein. A single laser system can be configured for both glaucoma treatment and treatment of refractive error for example. In another example, the single laser system can be configured to apply annular patterns of laser energy adjacent Schlemm's canal to dilate Schlemm's canal, as described in incorporated WO 2018/049246 or US 2018/0207029, and/or open an iridocorneal angle of the eye by delivering energy ab externo to a plurality of treatment locations at least about 2 mm radially outward from a limbus of the eye to treat glaucoma of the eye as disclosed in PCT/US2018/052261 that is incorporated by reference.

In some examples the processor can be configured with instructions to apply energy with patterns, amounts, intensities, and durations as described herein to treat the glaucoma. In addition to treating the collector channels/ostia, the energy source and instructions can be configured to apply a generally annular pattern of energy to the eye near Schlemm's canal, for example as in US 2018/0207029 and PCT/US2018/052261 that is incorporated by reference. The generally annular pattern can be aligned to the eye with reference to the limbus, which is located at the corneal/scleral junction near Schlemm's canal. The location of Schlemm's canal with respect to the limbus may vary systematically with age and/or IOP. For example, Schlemm's canal may be further away from the limbus in younger eyes than in older eyes. Schlemm's canal may be further away from the limbus in patients with increased IOP compared to patients with normal IOP. Such variations may be taken into account when patterning treatment. For example, treatment may be patterned further out from the limbus in an older patient than in a younger patient to account for the difference in location of Schlemm's canal with reference to the limbus.

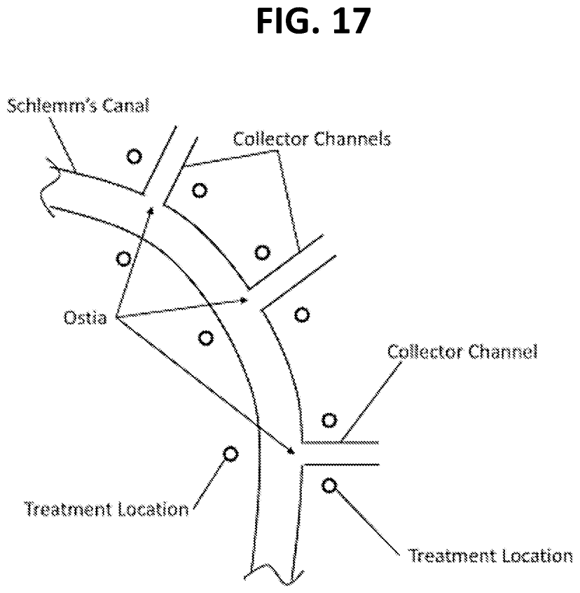

When treating the collector channels and ostia (FIG. 17), the plurality of treatment locations may be spaced from but juxtaposed within 1 mm of the collector channels. In some examples the treatment locations may also be spaced posteriorly from Schlemm's Canal by 1-2 mm, or less than 2 mm. The plurality of treatment locations extend in a first treatment pattern on a first side of the each of the collector channels and a second treatment pattern on a second side of said each of the collector channels opposite the first side in order stretch tissue between the first treatment pattern and the second treatment pattern to dilate said each of the collector channels to increase flow of the collector channels of the eye. The first treatment pattern may be located at a first angle relative to the optical axis of the eye and the second treatment pattern may be located at a second angle relative to the optical axis of the eye. The treatment locations may be in pairs (treatment locations a and b), one on each side of the collector channel as shown in FIG. 17. Collector channels are spaced irregularly around the limbus hence the pair of treatment locations are not equally spaced from other pairs of treatment locations around the eye.

The plurality of treatment locations may comprise one or more of at least one treatment location on a lateral side of an individual collector channel, at least one treatment location on an anterior side of said individual collector channel, at least one treatment location on a posterior side of said individual collector channel, at least one treatment location on an anterior side of said individual collector channel, at least one treatment location opposed from an ostia of said individual collector channel and adjacent the Schlemm's canal, or at least one treatment location within 1 mm of said ostia of said individual collector channel In some examples, all the treatment locations are on the sclera and none are on the cornea. In other examples, there are more pairs inferiorly and nasally than superiorly and temporally. A first treatment pattern extends at least about 30 degrees around the optical axis of the eye and the second treatment pattern extends at least about 30 degrees around the optical axis of the eye. The first treatment pattern extends at least about 40 degrees around the optical axis of the eye and the second treatment pattern extends at least about 40 degrees around the optical axis of the eye.

The processor may be configured with instructions to repeatedly deliver the energy to each of the plurality of treatment locations with a time delay in order to fractionate delivery of energy to said each of the plurality of treatment locations. The time delay may be within a range from about 10 millisecond (ms) to about 60 (s) and optionally wherein the time delay may be within a range from about 100 ms to about 30 s and optionally within a range from about 500 ms to about 15 s and optionally within a range from about 1 s to about 10 s. The processor coupled to the energy source and the scanner may be configured with instructions to heat tissue at the plurality of treatment locations to a temperature within a range from 50 to 70° C., for example 50 to 60° C. a depth within a range from 50 to 400 μm at each of the plurality of treatment locations along the treatment pattern. The duration of treatment of the location, and the total energy delivered, is sufficient to cause the treatment location to contract or shrink without denaturation of the contracted tissue.

A majority of a treatment energy of the treatment pattern may be located within 0.75 mm of each of the collector channels. The plurality of treatment locations may comprise one or more of treatment locations on a superior-nasal quadrant of the eye, treatment locations on an inferior-nasal quadrant of the eye, treatment locations on a superior-temporal quadrant of the eye, or treatment locations on an inferior-temporal quadrant of the eye.

The plurality of treatment locations extends in a treatment pattern arranged to avoid heating tissue overlaying one or more of the Schlemm's canal or at least one of the collector channels to the Schlemm's canal. The plurality of treatment locations extends in a treatment pattern comprising one or more of a circular, oval, elliptical, egg-like, non-circular, non-elliptical, or asymmetrical shape pattern.

Without being bound by any particular theory, applying the treatment locations a and b on opposing sides of a collector channel induces stretching between the treatment locations a and b to dilate the collector channel that lies between treatment locations a and b.

The contact lens, heat sink, and/or cooling structure as described herein can be used to conduct heat to reduce heating, for example when the energy source comprises a light source such as a laser as described herein, in order to leave the epithelium substantially intact. The energy source can be applied at locations in order to shrink tissue near Schlemm's canal and provide dilation of Schlemm's canal, the trabecular meshwork, the iridocorneal angle, or the collector channels, and combinations thereof in order to increase aqueous outflow and reduce intraocular pressure. The combination of dilation of Schlemm's canal, the trabecular meshwork, collector channels, and/or iridocorneal angle are particularly advantageous to the treatment of glaucomas that may have multifactorial causes.

Scleral vacuoles can be formed by treating scleral tissue with treatment parameters as described herein. The scleral tissue may be treated with a generally annular pattern, for example a plurality of spaced apart rings in order to create or expand vacuoles for improved outflow through the sclera. Alternatively, the annular treatment pattern may comprise an annulus, or portion thereof, for example. The annular treatment pattern may comprise a plurality of overlapping rings or spots from individual laser pulses, for example. The combined systems and methods described may be used to treat all types of glaucoma.

Glaucoma treatment energy may comprise laser energy as described herein, for example, although other forms of energy can be used such as radiofrequency energy.

The eye includes a conjunctiva disposed over the sclera and the sclera is treated through the conjunctiva of the eye. Alternatively, the conjunctiva can be moved away from the sclera to treat an inner portion of the eye (e.g. sclera) located below the conjunctiva.

Imaging while Treating

In some examples herein, methods and systems can be used to image the tissue during treatment. Collector channel and ostia treatment systems may comprise an imaging apparatus such that the treatment can be combined with one or more imaging techniques, such as one or more of magnetic resonance (MR) imaging, ultrasound biomicroscopy (UBM), ultrasound (US) imaging, optical coherence tomography (OCT), optical coherence elastography (OCE), or US elastography transducer measurements. The imaging apparatus can be combined with the eye treatment, for example with simultaneous oblique trans-iridional imaging or on the coaxial therapeutic probe; and diagnostic images that are useful intra-operatively, for visualization as well as for feature/landmark tracking. Rapid real time MR images can be acquired when time-synchronized to treatment energy pulses with weighting motion gradients turned ON for greater cavitational sensitivity. MR/OCT/US guided treatment guidance can include one or more of pretreatment planning, image-based alignment and siting of the treatment energy source focus, real-time monitoring of treatment energy-tissue interactions, or real-time control of exposure and damage assessment.

Examples of treatment systems may include an imaging apparatus capable of determining collector channel and ostia location or other characteristics before, during, or after eye treatment, or some combination thereof. The treatment system may additionally or in combination comprise a mechanism for real-time temperature sensing, for example using temperature sensors (e.g., IR), or an OCT transducer, in order for real-time monitoring of laser- or HIFU-induced temperature changes or to provide for control of laser or HIFU exposure, respectively, to maintain or adjust temperature.

Motorized diagnostic imaging in sync with histotripsy patterning can be achieved in these configurations. For example, real-time imaging of treatment tissue may allow for user input to a grid of target regions, which may be larger than the area covered by a single treatment or include multiple areas not in direct contact with each other, for motorized control of multiple treatments over a larger area, allowing the user to avoid manual repositioning which may save time and prevent mistakes.

Imaging may be configured to occur simultaneously with treatment. A processor can be coupled to the ultrasound array and configured with instructions to scan the beam to a plurality of locations and image the tissue during treatment. The system may also comprise a display coupled to the processor that allows the user to see the tissue treated on the display and to plan the treatment. The images shown on the display can be provided in real time and can allow the operator to accurately align the tissue with the treatment and may allow the operator to visualize the treatment area, and other locations away from the treatment area. The imaging of the treatment area can be used to determine identify the target area on the screen and to program the treatment depth and location in response to the images shown on the display. The imaging can be used to visualize movement of ocular structures during treatment in order to detect beneficial treatment effects. The processor can be configured with instructions to treat the eye with a first wavelength of ultrasound and to image the eye with a second wavelength longer than the first wavelength. The processor may alternatively or in combination be configured with instructions to treat the eye with HIFU or laser energy and to image the eye with an embedded imaging apparatus, for example an optical coherence tomography (OCT) probe. The processor coupled to the array can be configured with instructions to provide both ultrasound wavelengths from the array. The imaging apparatus may provide additional tissue feedback data in real-time, for example temperature or elasticity.

The processor may be configured with instructions to determine one or more locations of the collector channels, and/or one or more locations of the ostia. In response to the determined locations the processor may be configured with instructions to determine a treatment pattern based on the one or more locations of the limbus and/or the one or more locations of Schlemm's canal. The treatment pattern may for example comprise a treatment pattern that straddles a plurality of collector channels with pairs of treatment locations. The processor may be configured to deliver shrinkage energy to the sclera, to urge tissue near the collector channel to move towards the treated tissue and dilate the collector channel as described herein.

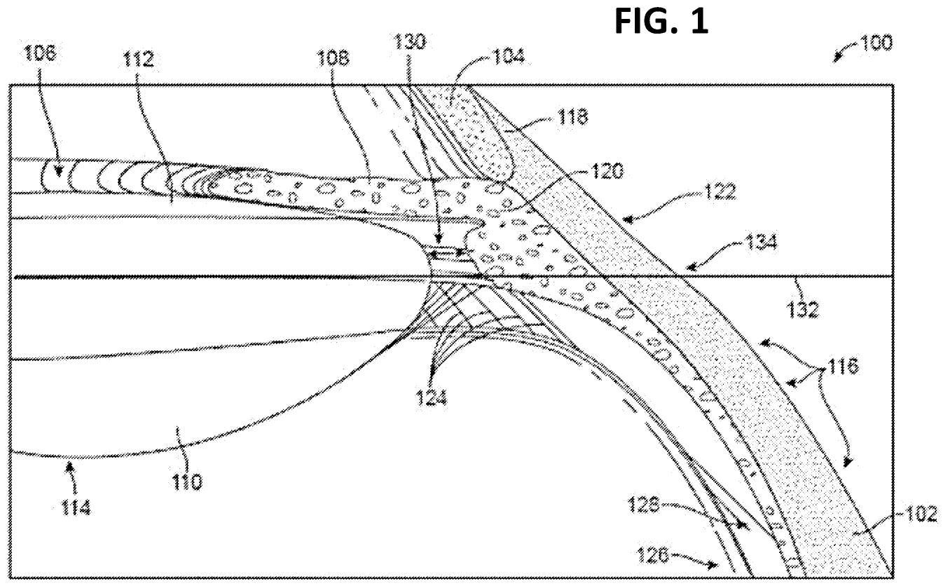

FIG. 1 illustrates an eye 100, in accordance with embodiments. The eye 100 includes a sclera 102, a cornea 104, a pupil 106, an iris 108, and a lens 110 within a lens capsule, the lens capsule including a lens capsule anterior surface 112 and a lens capsule posterior surface 114. The sclera is lined by a conjunctiva 116 and includes a sclera spur 118 adjacent the cornea 104. A ciliary body 120 is adjacent the ciliary body sclera region 122. The ciliary body 120 is connected to the lens 110 by vitreal zonules 124 and to the ora serrata 127 by the posterior vitreal zonules 128 (hereinafter “PVZ”). A circumlental space 130 (hereinafter “CLS”) is defined by the distance between the lens 110 and the ciliary body 120 along a lens equator plane 132, the lens equator plane 132 passing through an equatorial sclera region 134.

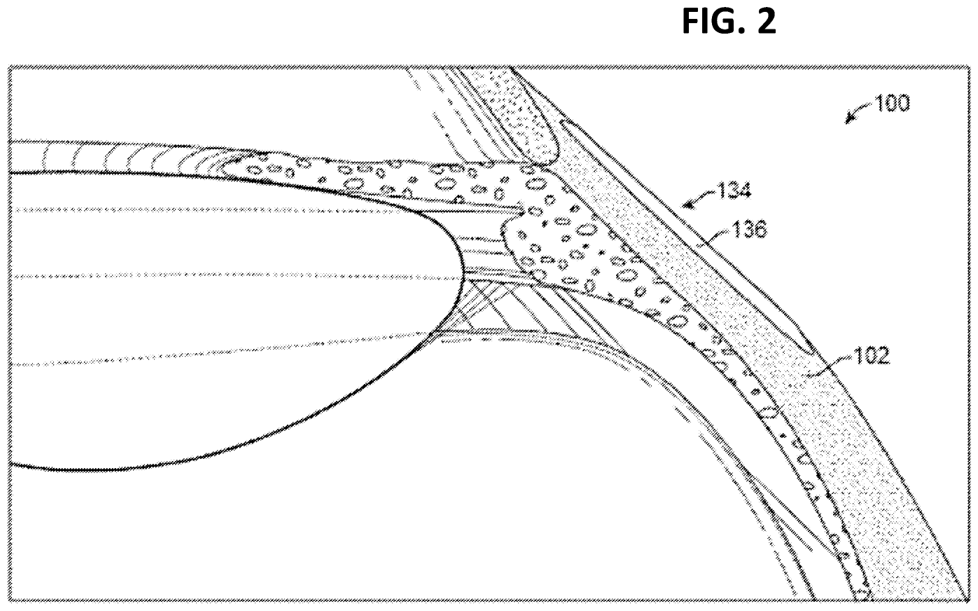

FIG. 2 illustrates stabilization of an eye 100 by cross-linking, in accordance with embodiments. The stabilized region 136 can be disposed in the outer portion of equatorial sclera region 134 of the sclera 102. Any suitable stabilization method, such as collagen cross-linking, can be used to stabilize the cross-linked region 136 in order to substantially maintain the outer profile of the sclera 102. In many embodiments, a cross-linking agent is applied to the sclera and allowed to infuse into the sclera at stabilized region 136. An energy source can be applied to the sclera to cross-link the sclera at stabilized region 136 with the cross-linking agent. The energy source can include a microelectrode array to generate a patterned cross-linked profile on the sclera. The energy can include one or more of thermal energy, radiofrequency (hereinafter “RF”) energy, electrical energy, microwave energy, light energy, or ultrasound energy.

In many embodiments, the cross-linking agent includes one or more of many known chemical photosensitizers in the presence of oxygen. Oxygen can be delivered to the stabilized region 136 of the sclera, concurrently with the cross-linking agent or separately. The cross-linking agent can be exposed to light energy when the cross-linking agent has been provided to the tissue, in order to provide cross-linking to a target depth of tissue stabilization. The light energy may include one or more of visible light energy, ultraviolet (hereinafter “UV”) light energy, or infrared (hereinafter “IR”) light energy. Alternatively, or combination, the cross-linking agent may include a chemical cross-linking agent. In many embodiments, the cross-linking agent includes one or more of the following: riboflavin, rose bengal, methylene blue, indocyanine green, genipin, threose, methylglyoxal, glyceraldehydes, aliphatic (3-nitro alcohols, black currant extract, or an analog of any of the above.

FIG. 3 illustrates common fluid outflow paths of the eye including the location of the limbus and Schlemm's canal. Glaucoma may be caused by obstruction to one or more fluid outflow paths. Aqueous humor is produced by the ciliary body processes and secreted into the posterior chamber. From there it flows through the narrow cleft between the anterior surface of the lens and the posterior surface of the iris, into the anterior chamber. The fluid may exit the anterior chamber via the trabecular outflow route and/or the uveoscleral outflow route into the anterior chamber angle (drainage canal) and out of the eye. The fluid may alternatively or in combination exit the anterior chamber through the iris surface and capillaries. In the trabecular outflow route, the fluid exits the anterior chamber and travels out of the eye via the trabecular meshwork. The fluid then drains directly into Schlemm's canal, an endothelial cell-lined channel at the limbus (where the cornea and sclera meet), or indirectly through collector channels and then into the episcleral venous system. In the uveoscleral outflow route, aqueous humor seeps through, around, and between tissues, including the supraciliary space, ciliary muscle, suprachoroidal space, choroidal vessels, emissarial canals, sclera, and lymphatic vessels, but does not have a well-defined structural pathway like the trabecular route. Blockage of one or more outflow pathways may increase intraocular pressure (IOP) and cause glaucoma. Reduction of IOP may treat glaucoma. Common mechanisms by which these mechanisms are blocked include closing of the anterior chamber angle, blockage of pores and/or vacuoles in Schlemm's canal, blockage (and/or collapse) or Schlemm's canal, blockage of uveo-sclera outflow (for example blockage of vacuoles or pores of the perilimbic sclera), inhibition of flow through the collector channels into the uveoscleral outflow, and any combination thereof.

Possible outcomes of the glaucoma treatment protocols described herein may include restoration of outflow through one or more of the outflow pathways. Treatment may be used to open the collector channels and/or ostia, to open a closed angle, dilate and/or stretch the trabecular meshwork, dilate and/or stretch Schlemm's canal, increase porosity and/or dilate vacuoles of the perilimbic sclera, or any combination thereof.

Treatment may be patterned or located so as to open a closed angle, open Schlemm's canal and/or the trabecular meshwork, open collector channels, change fluid bypass characteristics, stretch the trabecular meshwork, and/or improve the uveo-sclera outflow pathway. For example, angle closure may be treated with one or more paralimbal annulus, for example two or more paralimbal annuli. Schlemm's canal closure and/or trabecular meshwork closure may be treated with one or more juxtacanalicular annuli, for example two or more juxtacanalicular annuli, for example a first annulus radially inward from Schlemm's canal and a second annulus radially outward from Schlemm's canal. Increased porosity of the perilimbal sclera and/or dilation or vacuoles may include treatment to relax or stretch the supra-ciliary and/or sub-conjunctival sclera alone or in combination with treatment at the pars plana and/or pars plicata. Treatment to increase porosity may provide reduced intraocular pressure as a stand-alone treatment or in combination with other treatment methods or patterns as described herein. Increased porosity in the mid-stroma near the pars plana and/or pars plicata may for example enhance hydraulic conductivity/transport of the supra-choroidal, ciliary, and/or lymphatic fluid outflow pathways. Treatment may be patterned to flatten the iris in order to open closed angle. Treatment above the base of the iris root or the roof of the ciliary body may dilate Schlemm's canal and/or stretch the trabecular meshwork. In some cases, it may be beneficial to treat more than one region in a single patient. For example, treatment may be patterned so as to open angle, open Schlemm's canal and/or the trabecular meshwork, and increase porosity and/or dilate vacuoles of the perilimbic sclera. Treatments directed towards multiple indications may take around 1 minute to about 3 minutes to complete. Treatments directed towards dilating Schlemm's canal may be used to anteriorly expand the roof of Schlemm's canal by about 30 um to about 100 um. Changes in the cross-section of the trabecular meshwork and/or Schlemm's canal may cause scleral pores to expand and increase outflow, thereby improving glaucoma.

Treatment using the systems and methods described herein may treat glaucoma by improving homeostatic IOP mechanisms, so as to reduce intraocular pressure of the eye. For example, heating of one or more of the scleral, trabecular meshwork, or the ciliary body as described herein may induce one or more endogenous biological cellular cascades which may lead to improvements in outflow function. Without being bound by theory, heating of the target tissue with energy such as laser energy may stimulate heat shock protein (HSP) activation, which may lead to normalized cell functions, normalized cytokine expression, and improved auto-regulation of IOP. Such improved function may, for example, be related to opening of one or more of the collector channels, ostia of the collector channels, or the trabecular meshwork.

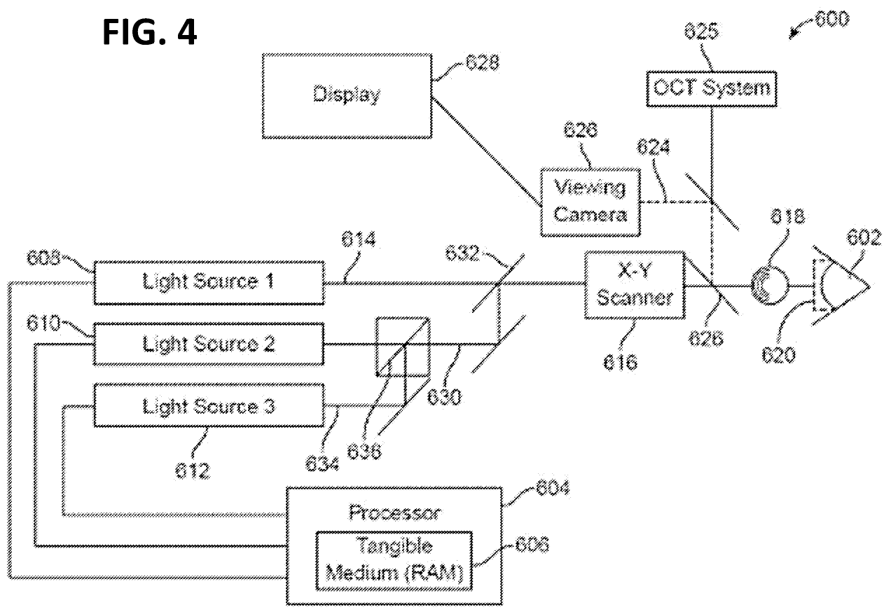

FIG. 4 illustrates a system 600 for treating an eye 602, in accordance with embodiments. The system 600 includes a processor 604 having a tangible medium 606 (e.g., a RAM). The processor 604 is operatively coupled to a first light source 608, an optional second light source 610, and an optional third light source 612. The first light source 608 emits a first beam of light 614 that is scanned by X-Y scanner 616 through an optional mask 618 and optional heat sink 620 onto the eye 602. The mirror 622 directs light energy from the eye 602 to a viewing camera 627 coupled to a display 628. An independent non-treatment light source for the optional viewing camera can be provided, for example. The mirror 622 may direct a portion of the light beam returning from eye 602 to the camera 627, for example. The second light source 610 emits a second beam of light 630 that is combined by a first beam combiner 632 with the first beam of light 614 prior to passing through X-Y scanner 616. The third light source 612 emits a third beam of light 634 that is combined by a second beam combiner 636 with the second beam of light 630 prior to passing through the first beam combiner 632. As shown, the beams are directed to the eye 602 in a generally direct, anterior-to-posterior direction, (e.g., parallel or close to parallel to an optical axis of the eye) though in some examples, other angles may be used, such as an oblique angle that produces an incidence from a peripheral position towards the eye that avoids impingement on the cornea.

The processor may be configured with one or more instructions to perform any of the methods and/or any one of the steps and sub-steps of the methods or treatments described herein. The processor may comprise memory having instructions to perform the method, and the processor may comprise a processor system configured to perform the method for example In many embodiments, the processor comprises array logic such as programmable array logic (“PAL”) configured to perform one or more steps of any of the methods or treatments described herein, for example.

The processor may comprise one or more instructions of a treatment program embodied on a tangible medium such as a computer memory or a gate array to execute one or more steps of a treatment method as disclosed herein. The processor may comprise instructions to treat a patient in accordance with embodiments described herein.

The processor may be configured with instructions to determine one or more locations of the limbus, and/or one or more locations of Schlemm's canal, and/or one or more locations of the collector channels and/or ostia. In response to the determined locations the processor may be configured with instructions to determine a treatment pattern based on the one or more locations of the collector channels and/or ostia. The treatment pattern may for example comprise a plurality of treatment locations on opposing sides of one or more collector channels. The processor may be configured to deliver shrinkage energy to the sclera to urge tissue adjacent the collector channels to move towards the treated tissue and dilate the collector channel as described herein.

The optical delivery system may comprise one or more of the first light source, second light source, third light source, X-Y scanner, optional mask, or a heat sink. The energy may be directed by the optical energy delivery system to the eye or a hand-held probe.

In many embodiments, the beams of light 614, 630, and 634 can be scanned onto the eye 602 at a specified X and Y position by the X-Y scanner 616 to treat the eye 602. The X-Y scanner can be configured to scan the combined light beams onto the eye 602 in a suitable treatment scan pattern, as previously described herein. An optional mask 618 can be used to mask the light applied to the eye 602, for example, to protect masked portions of the eye 602 while treating other portions as described herein. An optional heat-sink 620 can be placed on the eye 602 during treatment to avoid heating specified portions of the eye 602, as described herein.

The system 600 can be used to apply light energy to the eye 602 in accordance with any suitable treatment procedure, such as the embodiments described herein. In many embodiments, the first light beam 614 has a first wavelength, the second light beam 630 has a second wavelength, and the third light beam 634 has a third wavelength. Each wavelength can be a different wavelength of light. Alternatively, at least some of the wavelengths can be the same. For example, in accordance with the embodiments described herein, the first light beam 614 can have a wavelength suitable to: cross-link an outer portion of the eye 602 and shrink an inner portion of the eye 602; shrink the inner portion and cross-link the outer portion concurrently; shrink the inner portion after the outer portion has been cross-linked; or any suitable combinations thereof. Alternatively, the first light beam 614 can have a first wavelength suitable to cross-link the outer portion of the eye 602, as described herein, and the second light beam 630 can have a second wavelength suitable to shrink the inner portion of the eye 602, as described herein. The third light beam 634 can have a third wavelength suitable to soften a portion of the sclera of the eye 602, as described herein. Any suitable combination of wavelengths of light for applying any combination of the treatments described herein, concurrently or separately, can be used.

The processor can be coupled to each of the light sources to selectively irradiate the eye with light having wavelengths within a desired range of wavelengths. For example, the first light source can be configured to emit light energy having wavelengths in a range from about 1.9 to 2.1 μm, the 1/e attenuation depth can be in a range from about 200 to 300 μm, for example about 225 to 275 μm. The second light source can be configured to emit light energy having wavelength in a range from about 1.3 to 1.55 μm, the 1/e attenuation depth is within a range from about 350 to 450 μm. The processor can be programmed with instructions to irradiate tissue with light energy appropriate for the effect at the desired treatment location. For example, the light source emitting light energy in the range from 1.9 to 2.1 μm can be used to treat the cornea, and the second light source emitting light energy with wavelengths in the range from 1.3 to 1.55 μm can be used to irradiate the sclera. The software may comprise instructions of a treatment table so as to scan the laser beam to desired treatment locations as described herein.

The laser system 600 may comprise an OCT system 625, such as a commercially available OCT system. The OCT system may for example be a CASIA2 or CASIA SS-100 OCT scanner (TOMEY). The OCT system may for example be a commercially available OCT system such as one sold by Tomey, Heidelber, Visante, or Optovue. The OCT system can be coupled to the viewing optics and laser delivery system with a beam splitter 626. The viewing optics may for example comprise an operating microscope (such as one sold by Zeiss, Haag Streit, Leica, or Moller Weildel), a slit lamp, or other custom optics. The OCT system can be used to measure the eye in situ during treatment. For example, the OCT system can be used to generate OCT images as described herein in order to generate tomography of the eye to determine the location of target tissues, movement of target tissues, and stretching of target tissues as described herein. The OCT system 625 can be coupled to processor 604 and used to control the laser system with a feedback loop, for example.

The processor can be configured with instructions to scan the laser beam on the eye in accordance with the treatment patterns and parameters as described herein.

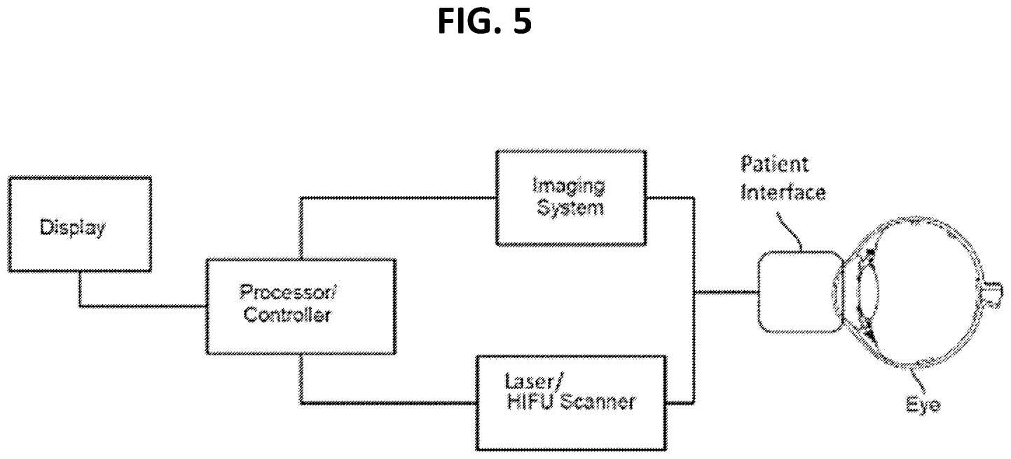

FIG. 5 shows another embodiment of a treatment system which may be used for any of the treatment methods described herein. The system may comprise a laser scanner (such as a 2D or 3D galvo-scanner) which directs and scans laser energy from a continuous wave or pulsed laser to one or more locations on the eye. The scanner may be coupled to a patient interface or patient coupling structure as described herein. The scanner may further be coupled to an imaging system, for example OCT or UBM, as described herein. The imaging system may be used to capture one or more images of the eye before, during, or after treatment as described herein. A processor or controller may be coupled to the energy source (e.g. HIFU transducer or laser) and the imaging system and be configured with instructions to scan the energy beam to a plurality of locations or in one or more patterns and image the tissue before, during, and/or after treatment. The system may also comprise a display coupled to the processor that allows the user to visualize the tissue prior to, before, or after treatment. The display may show images which allow the user to see the tissue treated and plan the treatment. Images shown on the display may be provided in real-time and can be used to prior to treatment to allow the user to align the tissue and/or select a treatment zone or pattern to target. Identified target treatment zones may be input by the user to program the treatment depth, location, and pattern in response to the images shown on the display. The imaging system can be used to visualize movement of ocular structures during treatment in order to detect beneficial treatment effects. The processor may be configured with instructions to treat the eye with laser energy and to image the eye with an embedded imaging apparatus, for example an OCT probe or US imager. The imaging apparatus may provide additional tissue feedback data in real-time, for example temperature or elasticity. The system as described herein may comprise an eye tracker as known to one in the art to generate real-time images of the eye in order to align or register the target treatment regions of the eye. Pre-treatment images can be measured and registered with real-time images obtained during treatment to track the location and orientation of the eye.

The glaucoma treatment systems described herein may simultaneously provide imaging guidance, quantitative characterization of the tissue (for example measuring mechanical properties such as elasticity), and/or perform therapeutic tasks.

Some embodiments may comprise two or more lasers. The processor may be configured with instructions to treat the eye with a first wavelength of light at a first location (or plurality of locations) and a second wavelength of light at a second location (or plurality of locations). The treatment system described herein may comprise one or more lasers within a range of about 488 nm to about 6 μm, for example about 810 nm, about 1.3 μm to about 2.5 μm, about 1.5 μm to about 2.4 μm, about 1.47 μm, about 1.95 μm, about 2.01 μm, about 2.1 μm, about 4 μm to about 7 μm, about 5 μm to about 7 μm, or about 6 μm. Wavelengths on the lower end of the spectrum, for example an 810 nm or 1.47 laser may be used to treat the sclera. A 1.47 μm laser may be about twice as tissue penetrating as a 2.01 μm laser when equidosed. Wavelengths on the upper end of the spectrum, for example within a range of about 4 μm to about 7 μm may be used to directly target collagen and/or protein. A 6 μm laser may be used to create scleral vacuoles for uveoscleral outflow enhancements for example. The system may optionally comprise a first laser with a first wavelength and a second laser with a second wavelength. The system may comprise a first laser with a first wavelength within a range of about 1.4 μm to about 1.6 μm and a second laser with a second wavelength within a range from about 1.9 μm to about 2.3 μm. The system may comprise a first laser with a first wavelength of about 1.47 μm and a second laser with a second wavelength of about 2.1 μm. The 1.47 μm laser for example may be used to treat scleral tissue (or other recalcitrant, thick, dense, or opaque tissue) with deeper penetrance than the 2.1 μm laser. The 2.1 μm laser may for example be used to treat corneal tissue. Alternatively, the 2.1 μm laser may be used to treat scleral tissue. In some instances, the sclera may be treated with both the 1.47 μm laser and the 2.01 μm laser at the same or different treatment locations, as the different wavelengths of light may produce different effects within the sclera which may be complimentary in designing a treatment plan. The processor may be configured with instructions to rapidly switch between the 1.47 μm and 2.1 μm lasers during treatment. In some embodiments, the system may comprise a first laser with a first wavelength of about 1.47 μm and a second laser with a second wavelength of about 1.95 μm and the processor may be configured with instructions to switch between the 1.47 μm and 1.95 μm lasers during treatment.

In some embodiments, the transducer array and the processor may be configured to provide a plurality of pulses to a plurality of separate treatment regions separated by a distance. A duty cycle of each of the plurality of separate treatment regions may comprise a duty cycle less than a duty cycle of the transducer array. The plurality of separate regions may comprise a first treatment region receiving a first plurality of pulses and a second treatment region receiving a second plurality of pulses, wherein the treatment alternates between the first plurality of pulses to the first region and the second plurality of pulses to the second region to decrease a duty cycle of each of the plurality of treatment regions relative to the duty cycle of the transducer array in order to decrease treatment time of the first region and the second region. The first treatment region may for example be a first annulus and the second treatment region may be a second annulus.

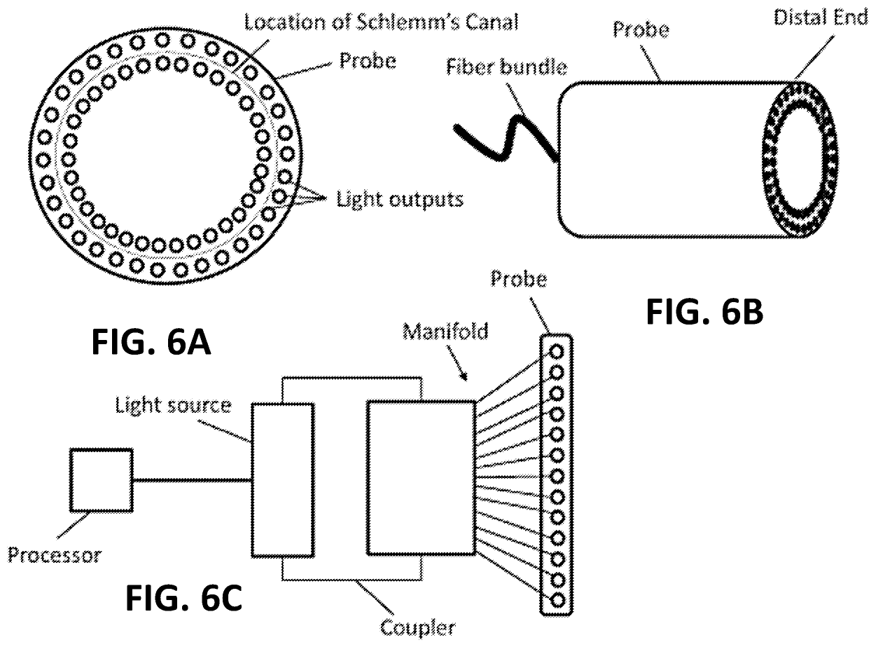

FIGS. 6A-6C show a handheld probe comprising a handpiece, in accordance with some embodiments. The handheld treatment probe may be used for any of the treatment methods and/or combined with components of any of the treatment systems described herein. FIG. 6A shows a plan view of the distal end of the handheld probe. FIG. 6B shows a perspective side view of the probe. FIG. 6C shows a schematic of the probe system, with a side plan view of the probe, coupled to a light source via a manifold in accordance with some embodiments.

The system may comprise a handheld probe which directs treatment energy to one or more locations on or inside the eye. In some instances, the distal end of the handheld probe may comprise a plurality of light outputs as shown in FIG. 6A. The light outputs may direct the treatment energy to one or more locations on or inside the eye. The light outputs may be oriented and/or spaced on the distal end of the handheld probe so as to target one or more region of the eye and/or avoid treatment in one or more region of the eye. For example, the light outputs may be arranged in so as to form two annuli on the distal end of the probe. The annuli may be spaced such that the outer annulus provides light energy to a portion of the eye that lies radially outward of Schlemm's canal and/or the limbus while the inner annulus provides light energy to a portion of the eye that lies radially inward of Schlemm's canal and/or the limbus. The dashed line in FIG. 6A represents an exemplary location of Schlemm's canal relative to the light outputs. In this way, the eye may be treated with the handheld probe in a juxtacanalicular manner as described herein. It will be understood by one of ordinary skill in the art that the light outputs may be arranged in any location and/or pattern on the distal end of the probe so as to provide treatment to the desired location(s) of the eye. For example, selected light outputs of the outer annulus shown in FIG. 6A are activated at desired locations so as to treat the sclera adjacent the collector channels and cause shrinkage of the scleral tissue to increase the diameter of the adjacent collector. The user may pattern the light outputs to avoid critical structures of the eye such as the vasculature.

The illustrated light outputs include a plurality of light sources such as laser diodes or light emitting diodes configured to emit light at a wavelength suitable for treating the eye as described herein. Alternatively, the plurality of light sources may comprise openings in a mask configured to transmit light to desired treatment locations and block light at other treatment locations. In some embodiments the treatment probe comprises a diffractive optic, an axicon or lenses, as is known to one of ordinary skill in the art, configured to delivery energy to the plurality of treatment locations. The light outputs may transmit light towards the eye from a light source external to the handheld probe, for example from a light source via a fiber bundle as shown in FIG. 6B and/or from a laser light source via a coupler and a manifold assembly as shown in FIG. 6C. In some instances, the light outputs may be controlled as one with a processor as described herein. In some instances, the light outputs may be individually and independently controlled, for example to adjust the treatment pattern delivered to a patient's eye. The light outputs may provide continuous or pulsed light energy to the treatment location. The light outputs may be configured to deliver light energy at the same wavelength or configured to deliver different wavelengths of light energy. For example, the outer annulus shown in FIG. 6A may comprise light at a wavelength of 1.48 μm to treat the sclera while the inner annulus may comprise light at a wavelength of 2.01 μm to treat the cornea. In some embodiments only the outer annulus delivers light to treat only the sclera and not the cornea.

The handheld probe may be configured to be directly coupled with the patient eye or it may be configured to be coupled to a patient interface or patient coupling structure as described herein.

It will be understood by one of ordinary skill in the art that the light outputs may be replaced by any source of treatment energy as described herein. For example, the light outputs may be replaced by radiofrequency electrodes or the like.

A system to treat glaucoma of an eye with the hand-held energy probe may comprise an energy source, such as one or more of the laser light sources as described herein. The handpiece comprising the treatment probe is coupled to the energy source. The handpiece comprises an eye contacting surface to couple to the eye on the distal end of the probe, and a plurality of energy releasing elements disposed at a plurality of locations to release energy to the eye at a plurality of treatment locations within 1 mm of the collector channels, for example in pairs of treatment locations on opposing sides of the collector channels. The distal end may comprise a concave shape such as a spherical shape or a conical shape to engage the eye near the collector channels. The energy releasing elements may comprise electrodes or light outputs such as ends of optical fibers. The plurality of locations corresponds to treatment locations located adjacent the collector channels.

The plurality of energy releasing elements may comprise a plurality of optical fibers and the energy source may comprise a laser.

Alternatively, or in combination, the plurality of energy releasing elements may comprise a plurality of electrodes and the energy source may comprise an electroporation energy source, a microwave energy source, a thermal energy source, an electrical energy source, an electrophoretic energy source, or a di-electrophoretic energy source.

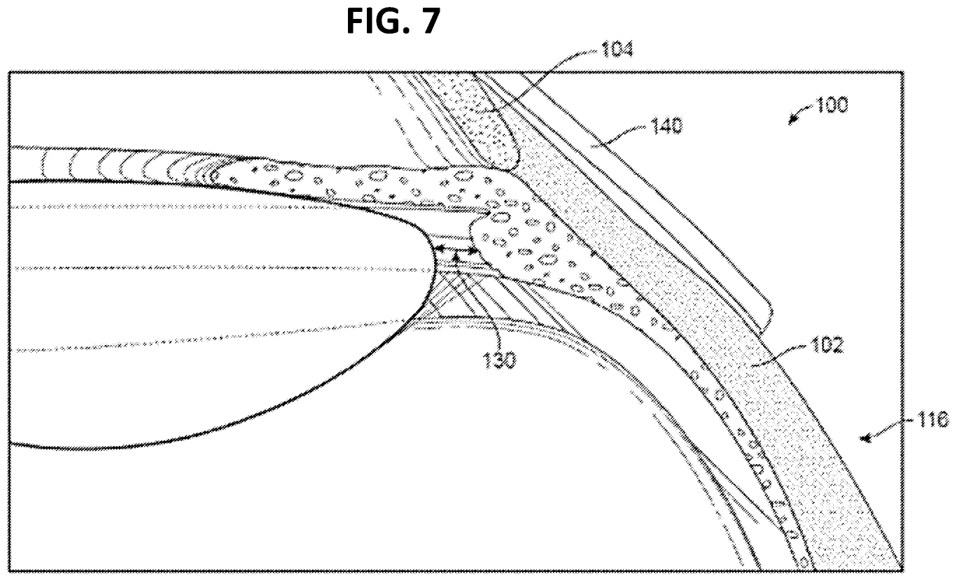

FIG. 7 illustrates a heat sink 140 placed over the eye 100 of FIG. 2 to treat glaucoma, in accordance with embodiments. The heat sink 140, for example a chilled contact lens, can be inserted over an outer portion of the eye 100 including the cornea 104, sclera 102, and conjunctiva 116, in order to conduct heat away from the outer portion of the eye 100 during the treatment procedure. The heat sink can be made of any suitable material. For example, the heat sink can include a material transmissive to wavelengths of light energy (e.g., sapphire or diamond-like carbon transmissive to certain IR wavelengths), so that the eye tissue beneath the heat sink can be heated with absorbed light energy.

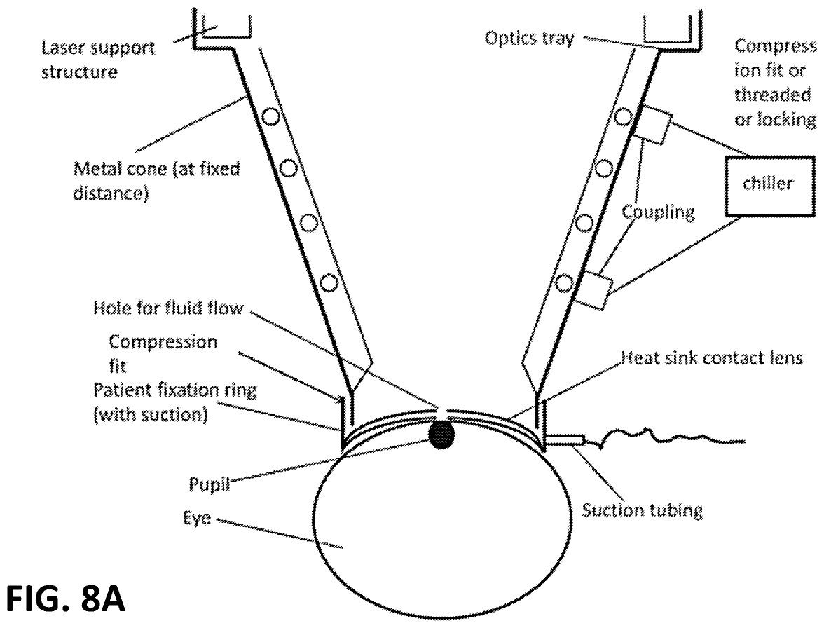

FIGS. 8A-8C show a structure for coupling an energy source to a surface of an eye. FIG. 8A shows a side view of a structure for coupling an energy source to a surface of an eye. FIG. 8B shows a side view of a structure for coupling an energy source to a surface of an eye. FIG. 8C shows a top view of a structure for coupling an energy source to a surface of an eye.

The structure may comprise a cone structure that is configured to remove heat from a surface of an eye. The cone may be composed of a material having a high thermal conductivity such as metal.

The cone may be coupled to a laser support structure and an optics tray. The laser support structure may support one or more laser sources, as described herein. The optics tray may support one or more optical components that direct one or more lasers to a surface of an eye, as described herein.

The cone may be coupled to a patient fixation ring. The patient fixation ring may be configured to form an air-tight seal with a surface of the cone. The patient fixation ring may be coupled to a surface of the cone using a compression fitting. The patient fixation ring may be configured to provide suction. For instance, the patient fixation ring may be coupled to suction tubing. The suction may be provided by connecting a syringe to the suction tubing and withdrawing the syringe. The suction tubing may comprise a vacuum pressure sensor. The pressure sensor may be used to determine that the coupling structure is properly connected to an eye.

The patient fixation ring may be coupled to a heat sink contact lens seated within the patient fixation ring. The heat sink contact lens may be composed of a material having a high thermal conductivity, such as sapphire, diamond or a diamond-like material. The heat sink contact lens may comprise a hole located at approximately the center of the heat-sink contact lens. The hole may allow for the flow of fluids (such as air) away from the eye. The heat sink contact lens may have an outer diameter of about 15 mm, 16 mm, 17 mm, 18 mm, 19 mm, or 20 mm or an outer diameter of less than 15 mm. The heat sink contact lens may have an outer diameter of greater than 20 mm. The heat sink contact lens may have a thickness of about 0.5 mm, 1 mm, or 1.5 mm. The heat sink contact lens may have a thickness of less than 0.5 mm. The heat sink contact lens may have a thickness of greater than 1.5 mm.

The cone may be positioned on a counter-weighted moveable arm such that no weight rests on the eye when the cone is docked to the patient fixation ring. The cone may have a fixed working distance such that the distance between the surface of the eye and the energy source may be constant between patients. The cone may be thermally controlled, for example with a fluid-based (such as water-based) heat exchanger or Peltier cooler, in order to help maintain the desired temperature of the patient interface and/or contact lens. Controlling the temperature of the cone may allow the preservation of tissues within the eye during an interaction with an energy source. For instance, cooling the cone may allow for the preservation of the epithelium during heating with a laser source.

In some embodiments, the cone may be thermally controlled using a thermoelectric cooler. The thermoelectric cooler may comprise a Peltier cooler placed in thermal connection with the heat sink lens. The Peltier cooler may be located on the counter-weighted moveable arms and may cool the cone to a temperature less than 37° C., less than 30° C., less than 25° C., less than 20° C., less than 15° C., less than 10° C., less than 5° C., or less than 0° C.

The system may be operated by optionally applying vacuum to the eye, aiming an illumination beam at the eye, and obtaining an OCT image of the eye. The OCT image may provide a baseline image of the eye prior to treatment. A treatment may be started once the heat sink contact lens has been secured in place by the vacuum. An OCT image may be obtained following treatment. The OCT image may be compared with the baseline image to obtain a precise measurement of changes induced by the treatment.

In some instances, medicaments may be added to the eye prior to and/or after treatment to further protect the corneal surface against thermal insult and/or stabilize treatment effects. Eye drops may contain medicaments which sequester and/or protect against collagen degradation and may be applied to the eye prior to and/or after treatment. In some instances, the medicaments may be collagen-sparing. The eye drops may contain medicaments such as hyaluronate or the like, polymers such as hydroxypropyl methylcellulose, and/or dissacharides optionally selected from the group consisting of Sucrose (table sugar, cane sugar, beet sugar, or saccharose), Lactulose, Lactose (milk sugar), Maltose (malt sugar), Trehalose, Cellobiose, Chitobiose, Kojibiose, Nigerose, Isomaltose, Trehalose (for example β,β-Trehalose or α,β-Trehalose), Sophorose, Laminaribiose, Gentiobiose, Turanose, Maltulose, Palatinose, Gentiobiulose, Mannobiose, Melibiose, Melibiulose, Rutinose, Rutinulose, Xylobiose, and any combination thereof.

In some instances, a topical anesthetic may be applied to the eye prior to or after treatment. Such anesthetics may include anesthetics with a tropane skeleton optionally selected from the group consisting of the Amylocaine, Articaine, Benzocaine, Bupivacaine, Butacaine, Carticaine, Chloroprocaine, Cinchocaine/Dibucaine, Cyclomethycaine, Etidocaine, Eucaine, Fomocaine[55], Fotocaine[55], Hexylcaine, Levobupivacaine, Lidocaine/Lignocaine, Mepivacaine, Meprylcaine/Oracaine, Metabutoxycaine, Phenacaine/Holocaine, Piperocaine, Pramocaine/Pramoxine, Prilocaine, Propoxycaine/Ravocaine, Procaine/Novocaine, Proparacaine/Alcaine, Quinisocaine, Risocaine, Ropivacaine, Tetracaine/Amethocaine, Trimecaine, and any combination thereof.

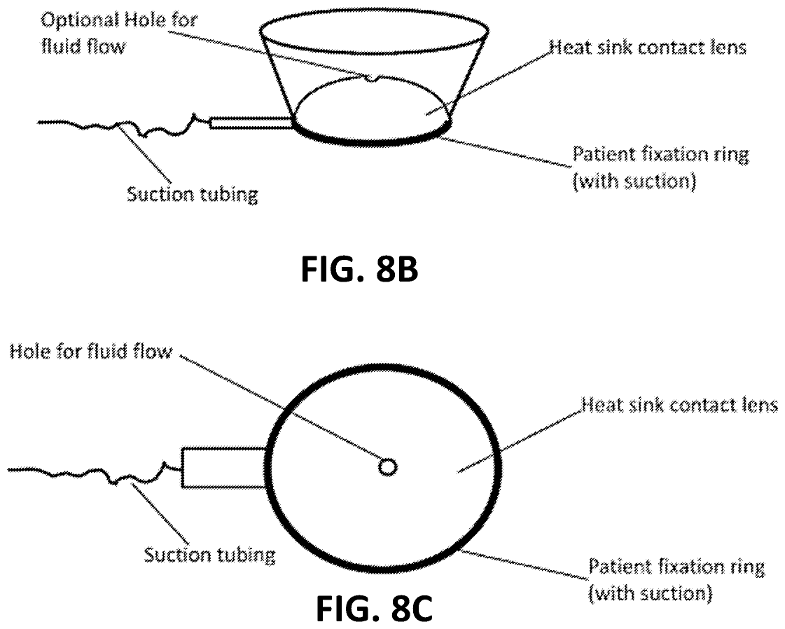

FIG. 9 shows temperature profiles of an eye treated with a laser beam with the eye coupled to a chilled lens based on computer modeling. This and other information about the use of the chilled contact lens are disclosed in US 2018/0207029 which is incorporated by reference.

FIGS. 9-14 show a treatment setup for collector channel and ostia treatment using ultrasound, for example high intensity focused ultrasound (HIFU), as an energy source. Additional information about the use of this ultrasound system is disclosed in US 2018/0207029 which is incorporated herein by reference.

### Treatment of Collector Channels and Collector Channel Ostia

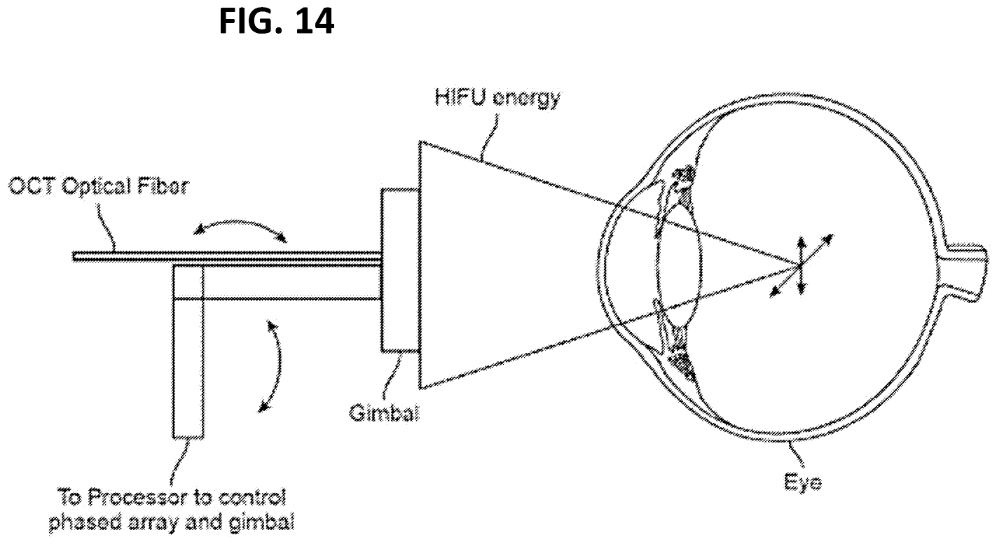

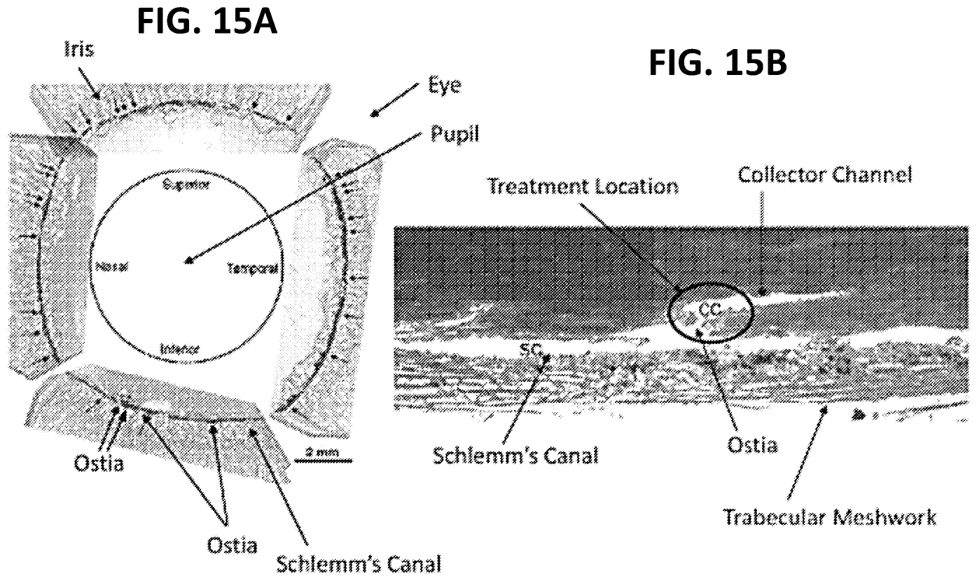

FIG. 15A is an anterior view of the eye, showing the pupil, the trabecular meshwork, the Schlemm's canal, collector channels coupled to the Schlemm's canal, and ostia of the collector channels. FIG. 15A highlights the distribution of collector channels in the normal eye. There is evidence that the majority of collector channels in the normal eye can be found in the inferior-nasal quadrant followed by the superotemporal quadrant. However, positions can vary from individual to individual. In various examples herein, collector channels can be identified through imaging, and treatment locations mapped based on the identifications. The orifice size of the collector channels (ostia) can have a wide range between 5 and 50 μm to as high as 70 μm. FIG. 15B shows a magnified section view of the Schlemm's Canal, an exemplary collector channel leading to the Schlemm's canal, and the collector channel ostia. FIG. 15B also shows a treatment location according to some embodiments herein; energy may be delivered to tissue within the treatment location to dilate the collector channel and/or the ostia to dilate said tissue structures to improve outflow. The treatment of the ostia and the collector channels can be combined with various treatments of the Schlemm's canal, including by way of example, annular treatments targeting Schlemm's canal, juxtacanalicular locations, etc., as well as with any additional examples described herein. For example, there is evidence that, even after removing, stripping, or otherwise adjusting the trabecular meshwork of the eye, a resistance to fluid outflow can remain. Selective treatment of the collector channels can allow fluid to pass out through the venous system of the eye, after having passed from the anterior chamber through the trabecular meshwork and into Schlemm's canal. Thus, treatment can be localized to regions near the collector channels and associated ostia as described herein, and in some examples additional portions of the eye can be targeted to improve outflow towards the collector channels.

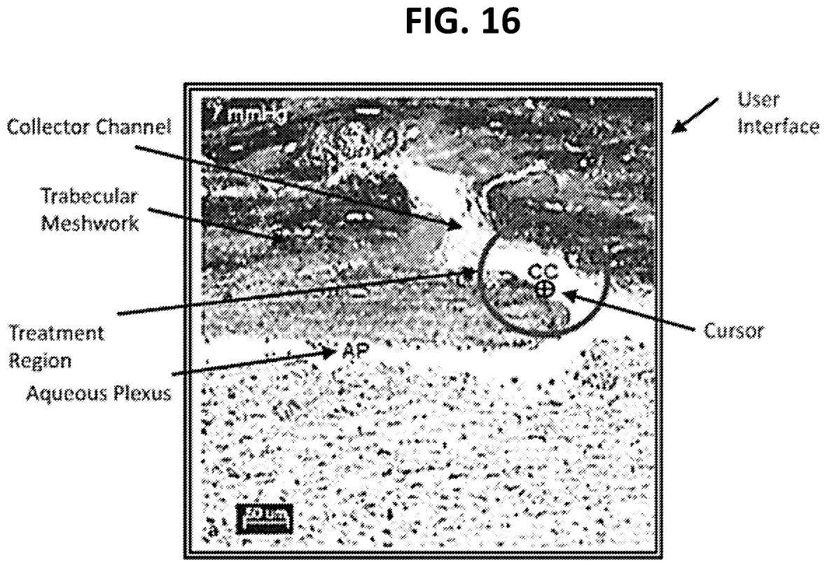

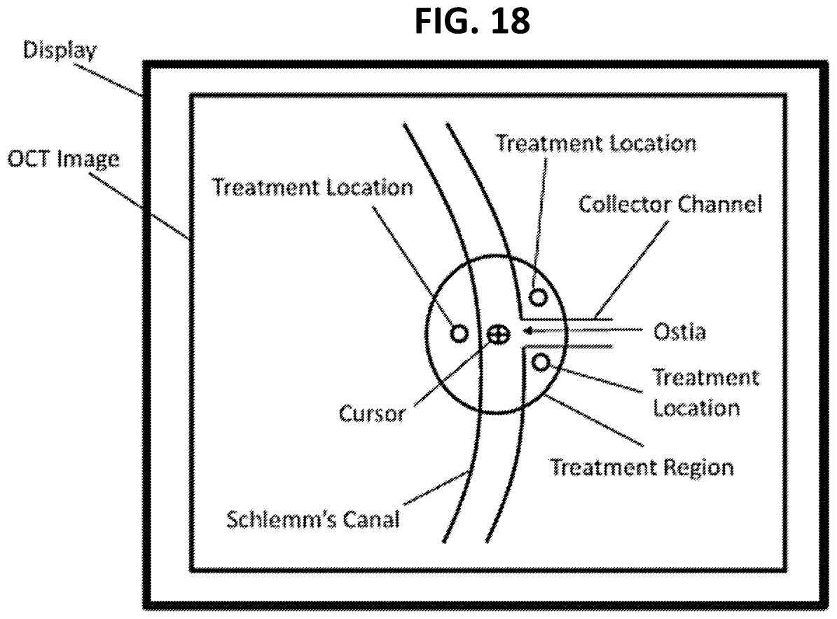

FIG. 16 and FIG. 18 shows exemplary user interfaces of a treatment system which may be used to plan a treatment of the human eye. (It will be appreciated that the methods and systems described herein can also be adapted to non-human eyes, such as cats, dogs, or other mammalia or vertebrates. The user interface may include a display such as a touch screen display for the user to view images of the subject and identify treatment locations. A scanner or other image source, such as an OCT scanner or high-resolution ultrasound scanner, may be used to obtain a sectional image of the eye, particularly a collector channel and the adjacent trabecular meshwork and aqueous plexus. For example, the systems and methods disclosed by Wang et al. in U.S. 2014/0236002 (herein incorporated by reference) can be used to image collector channels using OCT. The scanner or other image source may send the sectional image to the system processor which causes the image to be provided on the display. A cursor may further be provided with instructions to identify a treatment location at the collector channel and its ostia. As shown in FIG. 16, a section image of the anatomy may be displayed, and the cursor may be positioned on the display at the collector channel to identify the tissue within a bounded region near the collector channel as the treatment region. The processor can be configured with instructions to determine the placement of the energy beam as described herein in response to the input treatment region coordinates on the eye and register the target region with the image and physical location of the eye as described herein. A circle, oval, or other visible feature may be overlaid the section image to show the treatment region. The cursor may be centered on the treatment region and the user interface may be provided with control(s) to shrink and expand the treatment region and/or re-locate the cursor with respect to the treatment region. As shown schematically in FIG. 18, various treatment locations may be identified within the treatment region, automatically, semi-automatically, or manually with the identification of the treatment region. The user interfaces may be provided with control(s) to identify specific treatment locations and select treatment regimens for the treatment locations.

The energy system such as the laser system and ultrasound system as described herein can be configured to deliver an appropriately sized beam to the tissue at target location, and the beam size can be within a range from about 0.05 mm to about 1 mm, or 0.1 mm to about 0.5 mm, for example. The laser beam may comprise wavelengths that are absorbed by tissue as described herein, and the target tissue treated by thermal heating of tissue anterior to the target treatment region identified with the input treatment. For example, the laser system can be configured to scan the laser beam to a target location in response to an operating microscope image and the OCT image and treatment locations as described herein, and the laser programmed to transmit the beam through a covering lens to remove heat and into corneal, conjunctival or scleral tissue with some absorption in said tissue anterior to the treatment location identified in response to user input.

As disclosed herein, various treatment locations may be appropriate. FIG. 17 shows a schematic of the targeted anatomy and exemplary treatment locations. The treatment locations shown, for example, are radially adjacent each of the collector channels and opposite the ostia of the collector channels. Once identified, the treatment system may be used to apply energy to the tissue at the treatment locations. Treatment may shrink tissue in these locations and result in a dilation of the collector channels and/or their ostia, improving outflow. The energy applied may be any of the therapeutic energies disclosed herein such as laser energy. The parameters of the energy delivered may depend on the treatment selected for the treatment location and may be varied as discussed above.

A variety of other treatment locations and patterns may also be appropriate. The plurality of treatment locations may comprise juxtaposed locations located within 1 mm of the collector channels. The plurality of treatment locations may extend in a first treatment pattern on a first side of the each of the collector channels and a second treatment pattern on a second side of said each of the collector channels opposite the first side in order stretch tissue between the first treatment pattern and the second treatment pattern to produce a dilation of each of the collector channels to increase flow of the collector channels of the eye. The first treatment pattern may be located at a first angle relative to the optical axis of the eye and the second treatment pattern may be located at a second angle relative to the optical axis of the eye. In some examples, collector channel treatments can be used in conjunction with, or as an alternative to, well established treatment of the trabecular meshwork, such as trabeculoplasty, which is applied to the structure on the opposite side of Schlemm's canal.

The first treatment pattern may comprise a first plurality of spaced apart treatment patterns and the second treatment pattern may comprise a second plurality of spaced apart treatment patterns. The first plurality of spaced apart treatment patterns may comprise angularly separated spaced apart treatment patterns and the second plurality of treatment pattern may comprise angularly separated spaced apart treatment patterns. The first plurality of spaced apart treatment patterns may comprise radially separated spaced apart treatment patterns and the second plurality of treatment pattern may comprise radially separated spaced apart treatment patterns.

The plurality of treatment locations may comprise one or more of at least one treatment location on a lateral side of an individual collector channel, at least one treatment location on an anterior side of said individual collector channel, at least one treatment location on a posterior side of said individual collector channel, at least one treatment location on an anterior side of said individual collector channel, at least one treatment location opposed from an ostia of said individual collector channel and adjacent the Schlemm's canal, or at least one treatment location within 1 mm of said ostia of said individual collector channel In representative examples, multiple collector channels targeted for treatment, such as across an azimuth angle range of 90 degrees, 180 degrees, 360 degrees, etc., including in part or across an entire range of the superior nasal, inferior nasal, superior temporal, or inferior temporal quadrants. For example, a first treatment pattern range can extend at least about 30 degrees around the optical axis of the eye and a second treatment pattern range may extend at least about 30 degrees around the optical axis of the eye. In another example, a first treatment pattern extends at least about 40 degrees around the optical axis of the eye and a second treatment pattern may extend at least about 40 degrees around the optical axis of the eye. In further examples, a plurality of treatment locations are selected for collector channels and ostia across a range extending at least about 180 degrees around the optical axis of the eye. The plurality of treatment locations can be arranged to minimize shrinking tissue overlaying one or more of the collector channels or the Schlemm's canal.

The energy may be delivered to each of the plurality of treatment locations with a time delay in order to fractionate delivery of energy to the treatment locations to 400 microns at each of the plurality of treatment locations along the treatment pattern.

A majority of a treatment energy of the treatment pattern may be located within 0.75 mm of each of the collector channels. The plurality of treatment locations may comprise one or more of treatment locations on a superior-nasal quadrant of the eye, treatment locations on an inferior-nasal quadrant of the eye, treatment locations on a superior-temporal quadrant of the eye, or a treatment location on an inferior-temporal quadrant of the eye.

The plurality of treatment locations may extend in a treatment pattern arranged to avoid heating tissue overlaying one or more of the Schlemm's canal or at least one of the collector channels to the Schlemm's canal. The plurality of treatment locations may extend in a treatment pattern comprising one or more of a circular, oval, elliptical, egg-like, non-circular, non-elliptical, or asymmetrical shape pattern.

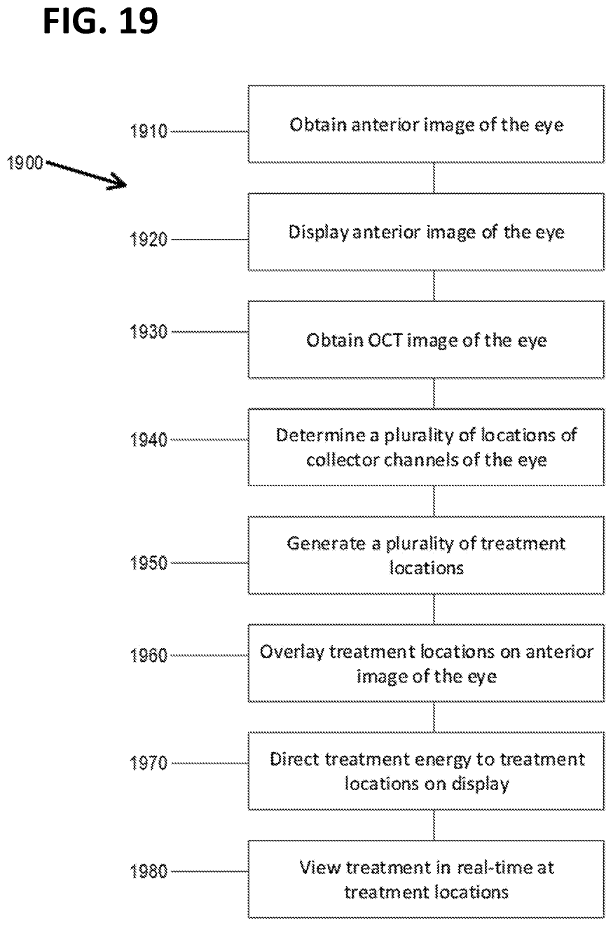

Referring to FIG. 19, a method 1900 for determining target treatment locations and treating the eye is shown. The method 1900 may use one or more of the systems described herein. In a first step 1910, an anterior image of the eye may be obtained by a camera or video recorder. In a second step 1920, the image of the eye may be displayed to a user as described herein. In a third step 1930, one or more OCT images of the eye may optionally be obtained. In a fourth step 1940, a plurality of locations of collector channels coupled to the Schlemm's canal may be determined from the anterior image of the eye, the one or more OCT images of the eye, or any combination thereof. The plurality of locations of the collector channels may be estimated manually by the user or automatically by the processor. The plurality of collector channel locations may optionally be registered with a corresponding plurality of anterior image locations. In a fifth step 1900, a plurality of treatment locations for the eye may be determined in response to the plurality of locations of the collector channels. The plurality of treatment locations may be determined manually by the user or automatically by the processor. In a sixth step 1900, the treatment locations may be overlaid onto the anterior image shown on the display. The treatment locations may optionally be adjusted or approved by the user. In a seventh step 1900, treatment energy may be directed to the treatment locations displayed on the image by an energy source and scanner as described herein. In an eighth step 5800, the treatment may be viewed in real-time at the treatment locations in order to adjust or halt treatment if movement of the eye occurs.

In representative examples, a processor may be provided for active assessment and/or control of treatment, such as for one or more of imaging eyes and collector channels, determining collector channel locations and treatment locations, or commanding energy source scanning to the treatment locations. The processor may be configured with instructions for perform a series of steps illustrated in FIG. 19 and others as described herein. In some instances, the processor may provide instructions to obtain an anterior image of the eye. For example, the anterior image of the eye may be obtained with a camera with aid of the processor. In some instances, the processor may be configured with instructions for receiving an anterior image of the eye.

In some instances, the processor may provide instructions to display the anterior image of the eye. In some instances, the processor may provide instructions to obtain OCT image(s) of the eye. In some instances, the processor may provide instructions to determine a plurality of locations of the collector channels of the eye. The processor may estimate in some instances the plurality of collector channel locations in response to the anterior image of the eye. Alternatively, or in addition, the processor may estimate the plurality of collector channel locations in response to the plurality of OCT images of the eye.

In some instances, the processor may be configured with instructions to generate a plurality of treatment locations. Optionally, the processor may be configured with instructions to generate the plurality of treatment locations for the eye in response to the plurality of collector channel locations.

In some instances, the processor may provide instructions to overlay treatment locations on the anterior image of the eye, for example, as in FIGS. 16 and 18. The processor may be configured with instructions to overlay the plurality of treatment locations and the plurality of collector channel locations on the anterior image of the eye. Optionally, the processor may be further configured to register the plurality of locations of collector channels with a corresponding plurality of anterior image locations. In some embodiments, the processor is configured with instructions to treat near the ostia with regions of the eye along Schlemm's between ostia substantially untreated in order to decrease the treatment time, and the untreated distance along Schlemm's canal between treatment locations can extend a distance of at least about 0.2 mm, for example from about 0.2 mm about 10 mm, or from 0.5 mm to about 5 mm.

In some instances, the processor may provide instructions to direct treatment energy to treatment locations on the display. In some instances, the processor may be configured with instructions to alternate treatment at a first plurality of treatment locations with treatment at a second plurality of treatment locations as described herein. Optionally, the processor may be configured with instructions to generate a third plurality of treatment locations located radially outward from the second plurality of treatment locations to generate vacuoles or increase a size of vacuoles in a sclera of the eye as described herein.

Optionally, the processor may be configured with instructions to generate a treatment table. The treatment table may comprise a plurality of coordinate reference locations corresponding to the plurality of treatment locations overlaid on the anterior image. Optionally, the energy source directed to the eye may comprise a pulsed energy source wherein each of the plurality of coordinate references corresponds to a pulse from an energy source.

In some instances, the processor may provide instructions to display treatment in real-time at the treatment locations.

Although the steps described above show a method of acquiring an image of an eye and treating the tissue at a treatment region selected by a user, one of ordinary skill in the art will recognize many variations based on the teachings described herein. The steps may be completed in a different order. Steps may be added or deleted. Some of the steps may comprise sub-steps. Many of the steps may be repeated as often as necessary to treat the tissue as desired. In some embodiments, a processor is configured to perform one or more steps of a method as described herein. The processor can be coupled to one or more of many types of energy sources such as laser energy sources, ultrasound energy sources, for example.

Referring back to FIGS. 6A-6C, a handheld probe comprising a handpiece, in accordance with some embodiments, is shown. The system may comprise a handheld probe which directs treatment energy to one or more locations on or inside the eye. In some instances, the distal end of the handheld probe may comprise a plurality of light outputs (such as optical fibers with focusing or collimation optics) as shown in FIG. 9A. The light outputs may direct the treatment energy to one or more locations on or inside the eye. The light outputs may be oriented and/or spaced on the distal end of the handheld probe so as to target one or more region of the eye and/or avoid treatment in one or more region of the eye. For example, the light outputs may be arranged in to form two annuli on the distal end of the probe. The annuli may be spaced such that the outer annulus provides light energy to a portion of the eye that lies radially outward of Schlemm's canal and/or the limbus while the inner annulus provides light energy to a portion of the eye that lies radially inward of Schlemm's canal and/or the limbus. To treat tissue adjacent the collector channels coupled to the Schlemm's canal, the light outputs, usually of the outer annulus, may be aligned to be radially adjacent (e.g., to the left and/or right of) individual collector channels, and only the light outputs aligned as such may be selected for use, while the remaining light outputs are selected so as to not delivery energy. Alternatively, or in combination, the light outputs, usually of the inner annulus, may be aligned to target tissue adjacent Schlemm's canal and anterior to the ostia of the collector channels. The light outputs which are aligned as desired may be selected for energy delivery while the remaining light outputs are selected to not delivery energy. The dashed line in FIG. 6A represents an exemplary location of Schlemm's canal relative to the light outputs. In this way, the tissue locations juxtaposed along the collector channels of the eye may be treated with the handheld probe in the manners as described herein. It will be understood by one of ordinary skill in the art that the light outputs may be arranged in any location and/or pattern on the distal end of the probe so as to provide treatment to the desired location(s) of the eye. For example, alternatively or in combination, an annulus of light outputs may be provided radially outward of the outer annulus shown in FIG. 9A at a desired location so as to treat the sclera to generate pores or vacuoles as described herein. Alternatively, or in combination, the user may pattern the light outputs to avoid critical structures of the eye such as the vasculature. In some examples, one or more aiming beams can be directed through the light outputs (e.g., at a different wavelength) so that positions of treating beams emitted from the light outputs can be aligned and observed before, during, or after treatment.

A system to treat glaucoma of an eye with the hand-held energy probe may comprise an energy source, such as one or more of the laser light sources as described herein. The handpiece comprising the treatment probe is coupled to the energy source. The handpiece comprises an eye contacting surface to couple to the eye on the distal end of the probe, and a plurality of energy releasing elements disposed at a plurality of locations to release energy to the eye at a plurality of treatment locations within 1 mm of a collector channel or collector channel ostia coupled to a Schlemm's canal of the eye. The distal end may comprise a concave shape such as a spherical shape or a conical shape to engage the eye near the Schlemm's canal. The energy releasing elements may comprise electrodes or light outputs such as ends of optical fibers. The plurality of locations corresponds to treatment locations located radially inward from the Schlemm's canal toward an optical axis of the eye and/or radially outward from the Schlemm's away from the optical axis of the eye or the center of the cornea as measured along an exterior surface of the eye. The variety of treatment locations within 1 mm of a collector channel or collector channel ostia coupled to the Schlemm's canal are further described herein.

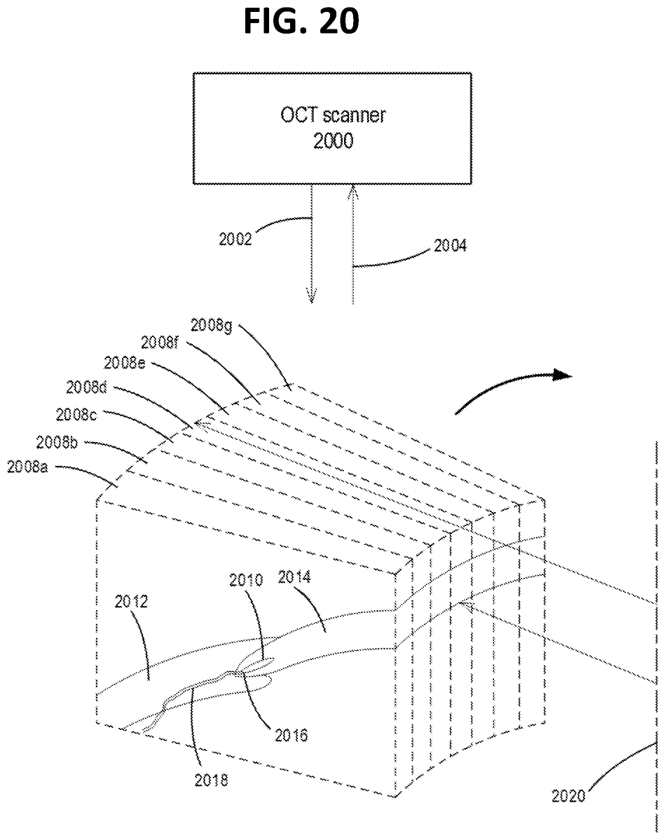

FIG. 20 shows an OCT scanner 2000 directing an OCT scan beam 2002 and receiving an OCT return beam 2004 from a portion of an eye 2006. OCT image slices 2008a-2008g are obtained for a portion of the eye 2006 that includes the tissue region proximate Schlemm's canal 2010, including the sclera 2012, cornea 2014, and also ostia 2016 and collector channels 2018. As shown, the image slices 2008a-2008g extend radially outward from an optical axis 2020 of the eye 2004, and are obtained azimuthally about the optical axis 2020, but other slice configurations are possible, such as annular, Cartesian slices, etc. The collector channel 2018 extends a substantial length in the image 2008a, though partial lengths can be observed depending on the slice, slice configuration, etc. Individual collector channels can be tracked over multiple slices, and various anatomical components identified, including hinged scleral flaps and cylindrical attachment structures proximate the ostia. In some examples, dye or other imaging aid can be administered to improve imaging contrast between the collector channel 2018 and surrounding tissue. The OCT scanner 2000 can collect images before, during, or after treatment, and in some examples, collector channel and ostia identification is achieved by comparing variations in images over time, such as a periodic collector channel dilation based on pulse or blood flow, or an applied pressure to Schlemm's canal.

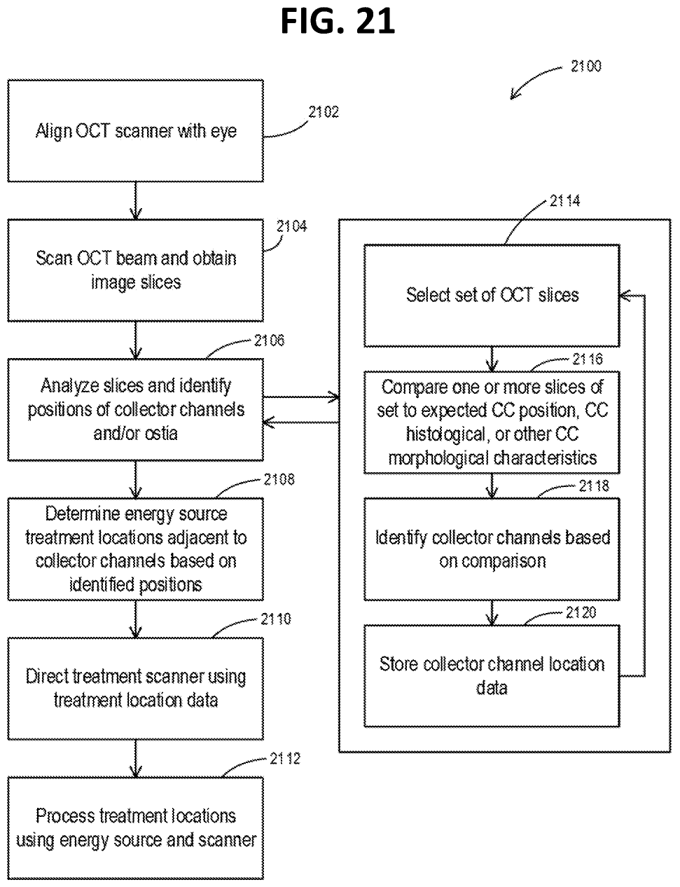

FIG. 21 is an example collector channel identification and treatment method 2100. At 2102 an optical coherence tomography (OCT) scanner, camera, or other imaging device is aligned with an eye that will receive treatment. For OCT devices, at 2104 an OCT beam is emitted and scanned across the eye to obtain OCT image slices. The OCT image slices are analyzed at 2106 and positions are identified for collector channels and/or ostia coupling the collector channels to Schlemm's canal. In some examples, anterior images are produced, and collector channels identified from the images, rather than using OCT image slices. At 2108, energy source treatment locations are determined based on the identified positions of the collector channels. In representative examples, energy source beams (e.g., laser, HIFU, etc.) are centered at positions adjacent to collector channels, such as rotationally (azimuthally) on either side of a collector channel, anterior and posterior of the collector channel, etc. In scanning-based examples, the treatment locations can be selected based on a predetermined spacing from the collector channel and ostium, such as 100 μm from the collector channel and 100 μm from the Schlemm's canal or ostium. Other distances can be used as well (e.g., 150 μm, 500 μm, 1 mm, etc.) and the distance away from Schlemm's canal can be different from the distance from the collector channel. After the treatment locations are determined, at 2110, a beam scanner can be commanded based on the treatment location data, and at 2112, the treatment beam is directed to the locations on the eye for treatment using the energy source and scanner. In some examples, the OCT scanner and the treatment energy beam and scanning source can be coupled to the eye along a common optical path (e.g., with a beam splitter). Additional examples can provide OCT scanning or imaging of the eye separate from treatment scanning.

As discussed herein, various beam parameters can be used and selected based on producing the dilation of the collector channels and ostia. Beam parameters typically vary in part based on the characteristics of the energy source. For example, laser beam parameters can include pulsed or continuous, and can include variation in wavelength, power, pulse energy, duty cycle, pulse repetition frequency, duty cycle, pulse duration, cross-sectional intensity profile, spot size, focus position, etc. Suitable wavelengths range from visible to far infrared, such as 532 nm (or shorter in selected examples, including ultraviolet), 790 nm, 810 nm, 1.030 μm, 1.064 μm, 1.3 μm to 1.55 μm, 1.9 μm to 2.3 μm, as well as longer wavelengths including 4 μm to 7 μm. Wavelength selection can also correlate with energy absorption and penetration depth in the tissue, allowing some wavelengths to shrink tissue to dilate collector channels more effectively than other wavelengths. In selected examples, the wavelength can be the same as a wavelength provided by as system configured to perform other procedures, such as laser trabeculoplasty. Beam power is generally selected in relation to pulse duration and total energy or fluence delivered to treatment areas. For example, continuous-wave (CW) laser sources at full (100%) duty cycle can be commanded to provide a power of typically less than 1 Watt for a predetermined duration, such as 10 ms, 100 ms, 500 ms, etc., bounded by respective rise and fall times. Continuous wave laser sources can also be electronically “chopped” to provide pulse-like characteristics with selected duty cycles (e.g., 1%, 10%, 50%, etc.). In some examples, a fractionated delivery can be provided by scanning the treatment beam to other areas of a treatment location or to another treatment location during zero or low-power periods of a duty cycle. Pulsed sources can have significantly larger peak-power with nanosecond pulses exceeding 1 kW. A 3 ns pulse with only 1.0 mJ energy has a peak-power of 333 KW. Other pulse durations including picosecond and femtosecond are possible, and microsecond pulses from laser diodes can be convenient. Pulses can be scanned to repeatedly impinge on a working treatment location or can be scanned to other treatment locations between pulses. The size of the focused spot of the laser beam is typically dependent on scanning and/or delivery optics and the laser source and wavelength. For example, single-mode beams can be produced with fiber lasers or coupling beams into single-mode fibers. Longer wavelengths increase the minimum spot size at the diffraction limit. Example spot sizes can range from 30 μm to 400 μm or larger. The total energy and fluence delivered is selected based on the aforementioned parameters and the desired shrinkage of the tissue proximate the ostia and collector channels and is typically less than a few Joules per treated eye. In some examples, less than 100 mJ is delivered to each treatment location. In a particular example, a localized elevation of temperature from 50° C. to 70° C. at the treatment locations is achieved with a delivery at a wavelength of 810 nm of an average power of 200 mW to 1400 mW (e.g., continuously at low power, or pulsed at higher power for various durations with one or more pulses) for between 0.1 s and 0.2 s with a laser spot size of 50 μm to 500 μm at a treatment location, followed by delivery to successive (typically to an adjacent “next” treatment location though jumps to more distance treatment locations are possible) treatment locations in a sequence with the same or similar parameters until the temperature elevation is realized at each of the treatment locations. Ultrasound or other energy sources can have different parameter sets as discussed above and in US 2018/0207029.

In general, treatment energy delivery from one or more laser sources (or ultrasonic, etc.) is configured to modify the sclera at the treatment locations adjacent to the collector channels to produce a scleral or collagen shrinkage, thereby increasing contractility to cause an opening of the collector channels and an improvement to aqueous outflow from Schlemm's canal. In scanning examples, one or more beam deflectors (such as rotatable galvanometer scan mirrors, acousto-optic deflectors) produce a change in a position of the beam at the target. Representative beam scanners include 3D scanners, which can vary an x-y position as well as a z-position of the beam, typically with an x-y scan mirror assembly and a lens arrangement provided with small adjustments to produce a commanded variation of a focal plane position (corresponding to the Z-scan direction). In some examples, positions of a plurality of light outputs can be fixed, such as the emitters of the example device shown in FIGS. 6A-6C. Positions of the light outputs can be fixed in relation to each other to provide treatment beam locations according to expected locations of collector channels and ostia. Devices with fixed emitters can be aligned with an eye and rotated to the correct position relative to the eye's optical axis and ocular quadrants.

The analysis and identification of collector channels and/or ostia at 2106 can be performed in various ways. In particular examples, at 2114, a set of OCT image slices obtained for an eye target is selected, such as a set of azimuthal slices that corresponds to an azimuthal range where collector channels are expected. In some examples, anterior images can be analyzed. Examples of collector channel imaging with OCT can be found in Wang et al. U.S. 2014/0236002. At 2116, one or more slices or anterior images are evaluated by comparing features with expected collector channel characteristics, such as probabilities of a collector channel at the azimuth position of the slice, shapes found in histological data, or other collector channel or ostia morphologies, including shape, color, or variations in shape or color over time or between slices. In some examples, a perfusion pressure can enhance feature detection, such as by producing a voxel variation between image slices at different times that can cause collector channels to become more discernible. In other examples, dyes may be used to increase a contrast between collector channels and adjacent tissue. At 2118, collector channels are successfully identified based on the evaluations at 2116, and at 2120 the collector channel location data is stored in a memory.

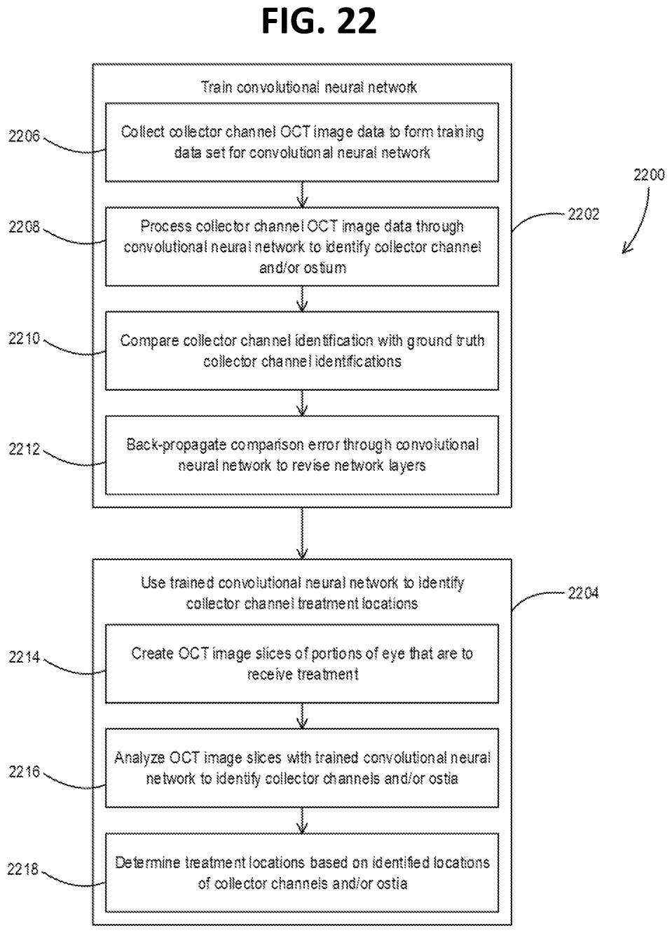

FIG. 22 is a machine learning based method 2200 that can be used in the process of identifying locations of collector channels and/or ostia so that related treatment locations can be determined. The method 2200 includes training a convolutional neural network (or constructing another probabilistic-based machine learning technique) at 2202 and using the trained convolutional neural network to identify collector channel treatment locations at 2204. For the training 2202, at 2206 CT image data or anterior image data of collector channels can be collected from eyes so as to form a training data set for the convolutional neural network. At 2208, the collector channel image data can be processed through the convolutional neural network to produce guesses for identifications of collector channels and/or ostia. At 2210, the identifications are compared with ground truth collector channel identifications and at 2212 comparison errors are back-propagated through the convolutional neural network to revise one or more network layers. Training can also be performed based on other image data, such as histological slice data. In use, at 2214, OCT image slices or other images of relevant portions of the eye proximate the collector channels and ostia are created for the eye that is to receive treatment. At 2216, the OCT image slices or other images are analyzed with the trained convolutional neural network to identify collector channels and/or ostia, and at 2218 treatment locations offset from collector channels are determined based on the identified locations of the collector channels and/or ostia.

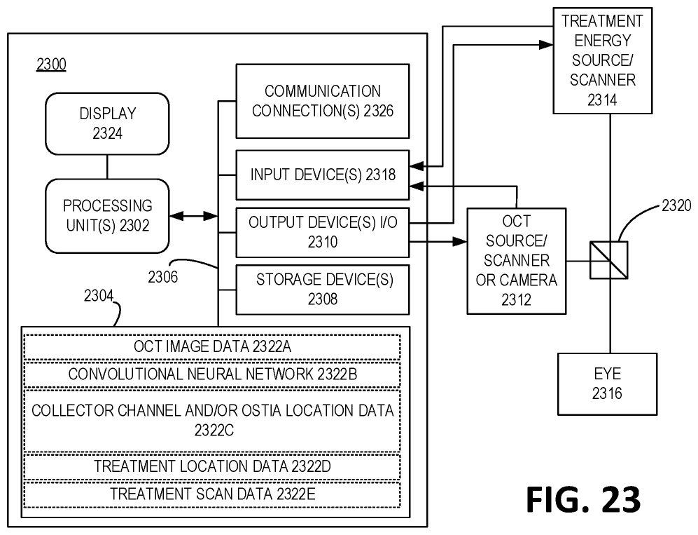

FIG. 23 and the following discussion are intended to provide a brief, general description of an exemplary computing environment in which the disclosed technology may be implemented. Computer-executable instructions, such as program modules, can be executed by a computing unit, dedicated processor, or other digital processing system or programmable logic device. Generally, program modules include routines, programs, objects, components, data structures, etc., that perform particular tasks or implement particular abstract data types. Moreover, the disclosed technology may be implemented with other computer system configurations, including hand-held devices, multiprocessor systems, microprocessor-based or programmable consumer electronics, network PCs, minicomputers, mainframe computers, dedicated processors, MCUs, PLCs, ASICs, FPGAs, CPLDs, systems on a chip, and the like. The disclosed technology may also be practiced in distributed computing environments where tasks are performed by remote processing devices that are linked through a communications network. In a distributed computing environment, program modules may be located in both local and remote memory storage devices.

FIG. 23 shows an example collector channel identification and treatment system that includes a computing device 2300 that includes one or more processing units 2302 (or processors), a memory 2304, and a system bus 2306 that couples various system components including the system memory 2304 to the one or more processing units 2302. The system bus 2306 may be any of several types of bus structures including a memory bus or memory controller, a peripheral bus, and a local bus using any of a variety of bus architectures. The memory 2304 can include various types, including volatile memory (e.g., registers, cache, RAM), non-volatile memory (e.g., ROM, EEPROM, flash memory, etc.), or a combination of volatile and non-volatile memory. The memory 2304 is generally accessible by the processing unit 2302 and can store software in the form computer-executable instructions that can be executed by the one or more processing units 2302 coupled to the memory 2304. In some examples, processing units can be configured based on RISC or CISC architectures, and can include one or more general purpose central processing units, application specific integrated circuits, graphics or co-processing units or other processors. In some examples, multiple core groupings of computing components can be distributed among system modules, and various modules of software can be implemented separately.

The computing device 2300 further includes one or more storage devices 2308 such as a hard disk drive for reading from and writing to a hard disk, a magnetic disk drive for reading from or writing to a removable magnetic disk, and an optical disk drive for reading from or writing to a removable optical disk (such as a CD-ROM or other optical media). Such storage devices can be connected to the system bus 2306 by a hard disk drive interface, a magnetic disk drive interface, and an optical drive interface, respectively. The drives and their associated computer-readable media provide nonvolatile storage of computer-readable instructions, data structures, program modules, and other data for the computing device 2300. Other types of non-transitory computer-readable media which can store data that is accessible by a PC, such as magnetic cassettes, flash memory cards, digital video disks, CDs, DVDs, RAMs, ROMs, and the like, may also be used in the computing environment. The storage 2308 can be removable or non-removable and can be used to store information in a non-transitory way and which can be accessed within the computing environment.

The computing device 2300 is coupled to an output device I/O 2310 so that suitable output signals (e.g., digital control voltage and/or current signals) are provided to OCT source/scanner 2312 and a treatment energy source/scanner 2314. The OCT source/scanner 2312 typically includes OCT illumination sources generating and directing imaging beams to an eye target 2316 to be imaged and treated. Input device I/O 2318 is coupled to the bus 2306 so that data signals and/or OCT image data and treatment location data can be stored in the memory 2304 and/or storage 2308 and/or processed with the processing unit 2302. In some examples, a beam splitter 2320 can be used to couple the OCT imaging and collector channel treatment beams along a common optical path.

In representative examples, the OCT image data received from the OCT source/scanner 2312 can be stored in a memory 2322A including location and/or time data. A deep learning network, such as a trained convolutional neural network, can be stored in a memory 2322B. The OCT image data in memory 2322A can be processed through the convolutional neural network in memory 2322B to determine collector channel and/or ostia position data that can be stored in a memory 2322C. Treatment locations can be determined from the collector channel and/or ostia position data in memory 2322C and stored in a memory 2322D and scan commands can be determined from the treatment locations and stored in a memory 2322E before sending to the treatment energy source/scanner 2314.

A number of program modules (or data) may be stored in the storage devices 2308 including an operating system, one or more application programs, other program modules, and program data. A user may enter commands and information into the computing device 2300 through one or more input devices such as a keyboard and a pointing device such as a mouse. Various other input devices can be used as well. These and other input devices are often connected to the one or more processing units 2302 through a serial port interface that is coupled to the system bus 2306, but may be connected by other interfaces such as a parallel port, game port, or universal serial bus (USB). In representative examples, the various routines, programs, and program modules can be automated so that collector channel eye treatment proceed with fewer operator or user steps or interventions. A display 2324 such as a monitor is also connected to the system bus 2306 via an interface, such as a video adapter. The display 2324 can be used to display OCT image slices, direct images of the eye surface, treatment positions overlaid on the eye surface, etc. Communication connections 2326 can be used to communicate with remote computers or other system components.

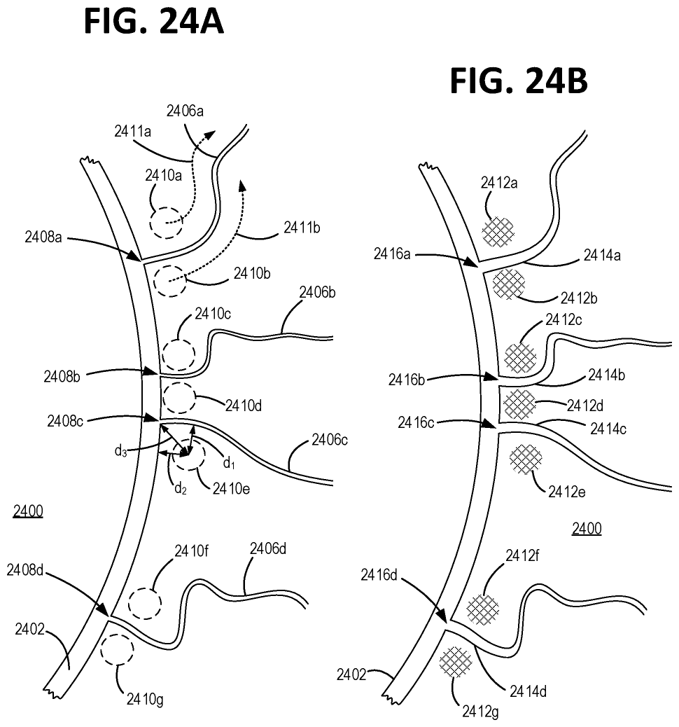

FIGS. 24A-24B show an example of an eye 2400 before (FIG. 24A) and after (FIG. 24B) treatment to improve outflow from Schlemm's canal 2402. Multiple constricted collector channels 2406a-2406d are arranged irregularly at different azimuth positions relative to an optical axis of the eye 2400. For example, any one collector channel (e.g., 2406b) may be spaced apart from an adjacent collector channel (e.g., 2406a) along one azimuth rotation direction differently from an adjacent collector channel (e.g., 2406c) along an opposite azimuth rotation direction, and the paths of the respective collector channels 2406a-2406d may include kinks and directional changes that are different or unique among the different collector channels. In some examples, selected collector channels may have anatomical characteristics (e.g., position, shape, quantity, pattern, etc.) that can be common across patient sets, and which can facilitate collector channel identification and/or treatment location selection for patient treatment. The constricted collector channels 2406a-2406d are coupled to the Schlemm's canal 2402 through respective constricted ostia 2408a-2408d. Treatment locations 2410a-2410g are determined based on the expected or identified positions of the collector channels 2406a-2406d and/or ostia 2408a-2408d. Multiple laser beam and scan parameters are selected for the laser beams directed to the treatment locations 2410a-2410d that produce a shrunken or scarred ocular tissue in respective treated regions 2412a-2412g. The shrinking of the tissue in the treated regions 2412a-2412g produce respective dilated collector channels 2414a-2414d and/or dilated ostia 2416a-2416d, which can improve fluid outflow from the Schlemm's canal 2402 and treat glaucoma or other related eye problems.

In representative examples, the circular shape and boundaries of the treatment locations 2410a-2410g correspond to the shape of laser spots used to treat the tissue, such as where intensity decreases to a full-width half maximum, 1/e, 1/e², etc. The treatment locations 2410a-2410g can form respective areas spaced apart in relation to adjacent collector channel, ostium, and/or Schlemm's canal, such as by distances d₁-d₃ for treatment location 2410e. Pairs of juxtaposed treatment locations can be formed, such as pair 2410a, 2410b, pair 2410c, 2410d, pair 2410d, 2410e (with 2410d forming an opposing treatment location for two separate pairs), and pair 2410f, 2410g that straddle respective collector channels 2406a-2406d. In representative examples, the distance d₂ from the center of a substantially circular treatment location to the posterior border of Schlemm's Canal is less than 0.5 mm or less than 1 mm In further examples, the distance d₂ is less than 2 mm In other examples the edge of the treatment location may be at least 0.5 mm or 1 mm from the posterior border of Schlemm's Canal. The parameters of the beam delivered to the treatment location (fluence, power, pulse duration, etc.) can also be selected in relation to the selected spacing of the treatment locations from the collector channels and Schlemm's canal, typically with reduced energies or less damage-prone beam parameters selected where shorter distances are selected or where tissue affected by treatment lies closer to the collector channels and/or Schlemm's canal. Typically, treatment location spacings are selected such that beam boundaries are spaced apart from Schlemm's canal and the collector channels. In some examples, the irregular paths of collector channels can be such that downstream posterior lengths of one or more collector channels may lie in a path of the principal axis of the treatment beam causing the anterior position of one or more the treatment locations to overlie the downstream posterior length. In some examples, the treatment location spacings are selected such that scarified or shrunken tissue is spaced apart from unaffected or less affected tissue, creating a less-affected tissue buffer between the treatment locations and Schlemm's canal and/or the collector channels. Beam boundaries and affected tissue boundaries can coincide or be different from each other depending upon beam parameter selection.

While circular spots are shown, other spot shapes can be formed, such as elliptical, square, rectangular, etc., and different spot intensity profiles can be selected (including top-hat shaped, donut shaped, Gaussian, etc.). Different treatment areas can be defined, including circular areas with a scanned spot to fill the area, as well as non-circular areas. In some examples, pairs of treatment locations can extend adjacently along paths 2411a, 2411b following the collector channels a predetermined distance away from the ostia, so as to extend a tubular dilation of the collector channel downstream of the ostia. Alternatively, or additionally, the extended beam paths or treatment location areas can further extend adjacent to the Schlemm's canal for a short distance to further dilate the adjacent ostium. The distances and/or shapes of each treatment location 2410a-2410d can be individually tailored to increase outflow. Spacings, area, and scan pattern can be configured in relation to tissue temperature or heat load to fractionate or reduce total energy delivery. While the treatment locations can be treated with laser beams scanned to the different treatment locations or scanned across areas within treatment locations, as discussed above, some example energy delivery sources can include fixed emitters, such as one or more arrays of optical fibers. In such examples, emitter ends can be positioned in relation to each other with a predetermined spacing to provide a selected pairing of treatment spots that straddle or are juxtaposed across one or more collector channels.

The energy (such as the laser) that irradiates the treatment location is sufficient to induce contraction of the irradiated tissue adjacent to the collector channels to exert tension on the tissue straddling the collector channel which in turn dilates the collector channel. For example, a laser irradiance (power over unit of area in W/cm²) and duration of irradiation may be selected to raise the temperature of the irradiated treatment location to 50-70° C., for example 50-70° C. Contraction of the tissue may be confirmed by direct visual observation of the treatment location, and/or dilation of the collector channel may be confirmed by the methods described herein for visualizing the collector channel.

In a specific example, a subject is selected for treatment based on clinical indications of glaucoma, such as increased intraocular pressure (IOP) measured by tonometry or applanation. Cupping of the optic disk or loss of visual field, even in the presence of normal IOP, may also be an indication for selecting the subject for treatment. The patient may have any type of glaucoma, such as primary open angle glaucoma or angle closure glaucoma, or a combination of glaucoma types. The disclosed methods for opening the collector channels are useful for treating a variety of forms of glaucoma by non-specifically increasing uveoscleral outflow and decreasing IOP.

Having described and illustrated the principles of the disclosed technology with reference to the illustrated embodiments, it will be recognized that the illustrated embodiments can be modified in arrangement and detail without departing from such principles. For instance, elements of the illustrated embodiments shown in software may be implemented in hardware and vice-versa. Also, the technologies from any example can be combined with the technologies described in any one or more of the other examples. It will be appreciated that procedures and functions such as those described with reference to the illustrated examples can be implemented in a single hardware or software module, or separate modules can be provided. The particular arrangements above are provided for convenient illustration, and other arrangements can be used. In view of the many possible embodiments to which the principles of the disclosed technology may be applied, it should be recognized that the illustrated embodiments are only representative examples and should not be taken as limiting the scope of the disclosure. Alternatives specifically addressed in these sections are merely exemplary and do not constitute all possible alternatives to the embodiments described herein. For instance, various components of systems described herein may be combined in function and use. We therefore claim all that comes within the scope of the appended claims.

1. A system for treating glaucoma of an eye, the system comprising:
a processor configured with instructions to:
receive input corresponding to a plurality of locations of collector channels coupled to a Schlemm's canal of the eye, and
generate a plurality of treatment locations for the eye in response to the plurality of locations, wherein the treatment locations are adjacent one or more collector channels and are spaced laterally from the collector channels by a distance of no more than 1 mm;
an energy source configured to generate energy to treat the eye; and
a scanner operably coupled to the energy source and the processor, the scanner configured to deliver the energy to the plurality of treatment locations to shrink tissue at the treatment locations and dilate the one or more of collector channels of the eye or ostia of the collector channels.

2. The system of claim 1, wherein the plurality of treatment locations comprise pairs of opposing treatment locations situated on opposite sides of each of the one or more collector channels.

3. The system of claim 2, wherein at least one of the pairs of treatment locations is positionally configured to stretch tissue between the opposing treatment locations of the at least one pair to provide the dilating of the one or more collector channels to increase flow of the collector channels of the eye;
wherein the processor is configured with instructions to identify the one or more collector channels or ostia from image data of the eye.

4-5. (canceled)

6. The system of claim 2, wherein the processor is configured with instructions to repeatedly deliver the energy to each of the plurality of treatment locations with a time delay in order to fractionate delivery of energy to each of the plurality of treatment locations;
wherein the time delay is within a range from about 10 millisecond (ms) to about 60 (s) and optionally wherein the time delay is within a range from about 100 ms to about 30 s.

7. (canceled)

8. The system of claim 2, wherein the processor coupled to the energy source and the scanner is configured with instructions to heat tissue at the plurality of treatment locations to a temperature within a range from 50 to 70 (° C.) at a depth within a range from 50 to 400 μm.

9. The system of claim 2, wherein the plurality of treatment locations extends in a treatment pattern arranged to avoid or reduce a heating of tissue overlaying one or more of the Schlemm's canal or at least one of the collector channels to the Schlemm's canal.

10. (canceled)

11. The system of claim 1, wherein the input comprises an input from a user of the system or an input from an imaging apparatus;
wherein the energy source comprises a laser having a wavelength within a range from about 0.8 to 2.3 μm;
wherein the energy source is configured to generate a treatment spot at or in the eye, the treatment spot being in a range of 30 to 500 μm across;
wherein the energy source is configured to generate an average power of 200 mW to 1400 mW.

12-19. (canceled)

20. A method for treating glaucoma of an eye, the method comprising:
determining a plurality of locations of collector channels coupled to a Schlemm's canal of the eye; and
delivering energy to a plurality of treatment locations adjacent to collector channels of the eye based on the plurality of locations, wherein the treatment locations are located within 1 mm laterally of the collector channels;
wherein the energy is delivered to the plurality of treatment locations to shrink tissue at the treatment locations to stretch one or more of at least one collector channel or an ostia of the at least one collector channel.

21. The method of claim 20, wherein the plurality of treatment locations comprises pairs of opposing treatment locations situated on opposite sides of each of the at least one collector channels to produce the stretching between opposing treatment locations to produce a dilation of the collector channel straddled by the opposing treatment locations or ostium of the straddled collector channel.

22. The method of claim 21, wherein at least one of the treatment locations corresponds to an opposing treatment location of two different pairs.

23. The method of claim 21, wherein the tissue is heated to a temperature within a range from 50 to 70° C. at a depth within a range from 50 to 400 μm at each of the treatment locations.

24. The method of claim 21, wherein the determining the locations includes identifying the collector channels from optical coherence tomography image data of the eye.

25. The method of claim 21, wherein the plurality of treatment locations is arranged to minimize shrinking of tissue overlaying one or more of the collector channels or the Schlemm's canal.

26. (canceled)

27. The method of claim 20, wherein the energy comprises energy from a laser having a wavelength within a range from about 0.8 to 2.3 μm;
wherein the energy is configured to generate a treatment spot in the eye, the treatment spot being in a range of 30 to 500 μm across.

28. (canceled)

29. An apparatus to treat glaucoma of an eye having a Schlemm's canal and collector channels coupled thereto, the apparatus comprising:
an energy source; and
a processor coupled to the energy source, wherein the processor is configured with instructions to direct energy in an irregular pattern associated with an irregular azimuthal positioning of the collector channels to shrink collagenous tissue near the collector channels coupled to the Schlemm's canal to dilate the collector channels.

30. (canceled)

31. The apparatus of claim 29, wherein the energy source comprises a laser having wavelength within a range from about 0.8 um to about 2.1 um;
wherein the laser is configured to deliver an amount of energy per unit time (power) to the eye within a range from about 50 mW to about 900 mW.

32-33. (canceled)

34. The apparatus of claim 29, wherein the processor is configured with instructions to apply a total amount of energy applied to the eye to treat glaucoma within a range from about 4 J to about 90 J, with a treatment time within a range from about 4 to 200 seconds, and configured with instructions to scan the energy source to the treatment locations on opposites side of the collector channels with a scan rate within a range from about 10 to 100 mm/second;
wherein the energy source comprises a laser, and wherein the laser comprises a cross-sectional beam spot size at or in the eye within a range from about 30 to 500 μm.

35-37. (canceled)

38. The apparatus of claim 29, wherein the processor is configured with instructions to estimate a plurality of collector channel locations in response to an anterior image of the eye or a plurality of optical coherence tomography (OCT) images of the eye, and to determine a plurality of treatment locations for the eye corresponding to the irregular pattern, based on the plurality of collector channel locations.

39. The apparatus of claim 29, wherein the processor is configured with instructions to identify collector channels and/or ostia from optical coherence tomography (OCT) images of the eye.

40. The apparatus of claim 29, wherein the energy source comprises an optical scanner, and the processor is configured with instructions to direct the energy to the treatment locations using the optical scanner.

41-66. (canceled)
